(19) United States
(12) Patent Application Publication
(10) Pub. No.: US 2006/0025928 A1
(43) Pub. Date: Feb. 2, 2006

(54) Hydropathy plots and fourier analysis with an ellipsoidal distance metric

(75) Inventors: Ajay Royyuru, Congers, NY (US); Benjamin David Silverman, Yorktown Heights, NY (US); Ruhong Zhou, Fort Lee, NJ (US)

Correspondence Address: Ryan, Mason & Lewis, LLP, Suite 205, 1300 Post Road, Fairfield, CT, 06824, US

(73) Assignee: International Business Machines Corporation, Armonk, NY (US)

(21) Appl. No.: 10/901,527

(22) Filed: Jul. 29, 2004

(51) Int. Cl. G06F 19/00; G01N 33/50; G01N 33/48

(52) U.S. Cl. 702/19

(57) ABSTRACT

Techniques for protein structure analysis are provided. In one aspect, a method of characterizing at least a portion of a protein structure comprising amino acid residues comprises the following steps. A set of values characterizing the protein structure are determined, wherein each value represents a distance from a center of the protein structure to a center of a given one or more of the amino acid residues. One or more other sets of values characterizing the hydrophobicity of the protein structure are obtained. A Fourier transform is performed on each of the sets of values to obtain transformed values sets. The transformed value sets are compared to correlate the hydrophobicity with the protein structure.

### FIELD OF THE INVENTION

The present invention relates to protein analysis and, more particularly, to techniques for characterizing protein structures.

### BACKGROUND OF THE INVENTION

Proteins are composed of a series of amino acid residues. There are 20 naturally occurring amino acid residues. The three-dimensional structure of a protein typically comprises a series of folded regions. When predicting the structure of a protein, researchers attempt to determine the amino acid spatial order and location in three-dimensional space. Obtaining the three-dimensional structure of a protein is important because protein function associated with the human body depends upon the particular protein structure.

Many proteins are globular and form in an aqueous environment. These globular proteins comprise hydrophobic amino acid residues that repel water, and hydrophilic amino acid residues that are attracted to water. When these proteins fold up, the hydrophobic amino acid residues are predominantly arranged in the non-aqueous center of the protein molecule and the hydrophilic amino acid residues are arranged on the aqueous protein surface. A protein formed in this manner will have a hydrophobic core and a hydrophilic exterior.

A number of previous studies have indicated that the hydrophobicity of sequences of amino acid residues is approximately random. However, the information that exists suggests that there is a relationship between hydrophobicity and protein structural features. Therefore, it would be desirable to correlate hydrophobicity and protein three-dimensional structure for protein study.

### SUMMARY OF THE INVENTION

The present invention provides techniques for protein structure analysis. In one aspect of the invention, a method of characterizing at least a portion of a protein structure comprising amino acid residues comprises the following steps. A set of values characterizing the protein structure are determined, wherein each value represents a distance from a center of the protein structure to a center of a given one or more of the amino acid residues. One or more other sets of values characterizing the hydrophobicity of the protein structure are obtained. A Fourier transform is performed on each of the sets of values to obtain transformed value sets. The transformed value sets are compared to correlate the hydrophobicity with the protein structure.

A more complete understanding of the present invention, as well as further features and advantages of the present invention, will be obtained by reference to the following detailed description and drawings.

### BRIEF DESCRIPTION OF THE DRAWINGS

FIG. 1 is a diagram illustrating an exemplary methodology for characterizing a protein structure according to an embodiment of the present invention;

FIG. 2 is a diagram illustrating an exemplary system for characterizing a protein structure according to an embodiment of the present invention;

FIG. 3 is a chart illustrating 30 protein database (PDB) globin proteins and their corresponding number of amino acid residues;

FIG. 4 is a collection of spectral graphs illustrating percent Fourier power amplitudes of the sequences of distance, exposure and hydrophobicity as a function of wavelength, for the protein 1HBG according to an embodiment of the present invention;

FIG. 5 is a chart illustrating correlation coefficients for thirty sample globin proteins according to an embodiment of the present invention;

FIG. 6 is a spectral graph illustrating the values of ellipsoidal distance, from the centroid of the protein, with the hydrophobicity of the smoothed and inverse transform of the prominent amplitudes for each amino acid residue along the sequence of the protein 1HBG according to an embodiment of the present invention;

FIG. 7 is a ribbon diagram representation of the protein 1HBG;

FIG. 8 is a spectral graph illustrating the inverse transform of the amplitudes of three hydrophobic periodicities superposed upon the original individual amino acid residue values of the hydrophobicity distribution according to an embodiment of the present invention;

FIG. 9 is a collection of spectral graphs illustrating percent Fourier power amplitudes of the sequences of distance, solvent exposure and hydrophobicity as a function of wavelength for the B chain of the protein 1HBH according to an embodiment of the present invention;

FIG. 10 is a spectral graph illustrating the values of ellipsoidal distance, from the centroid of the protein, with the hydrophobicity of the smoothed and inverse transform of the prominent amplitudes for each amino acid residue along the sequence of the B chain of the protein 1HBH according to an embodiment of the present invention;

FIG. 11 is a graph illustrating the spatial distribution of amino acid residue hydrophobicity from the interior environment of the protein 1HBG according to an embodiment of the present invention;

FIG. 12 is a chart illustrating Neumaier hydrophobicity scale values for the 20 naturally occurring amino acid residues;

FIG. 13 is a collection of spectral graphs illustrating percent Fourier power amplitudes of the sequences of distance, exposure and hydrophobicity over the range of wavelengths from two to five amino acid residues, for the protein 1HBG according to an embodiment of the present invention;

FIG. 14 is a collection of spectral graphs illustrating percent Fourier power amplitudes of the sequence of amino acid residue hydrophobicity over the range of wavelengths from two to five amino acid residues for the protein 1HBG, and for two randomized sequences according to an embodiment of the present invention;

FIG. 15 is a chart illustrating the PDB identification and number of amino acid residues for each of the immunoglobulin and cuprodoxin domains;

FIG. 16 is a chart illustrating correlation coefficients of the C1 and Plastocyanin/Azurin set of domains according to an embodiment of the present invention;

FIG. 17 is a collection of spectral graphs illustrating percent Fourier power amplitudes of the sequences of distance, exposure and hydrophobicity as a function of wavelength, for the B chain of the immunoglobulin C1 set domain protein, 1CD1 according to an embodiment of the present invention;

FIG. 18 is a spectral graph illustrating the values of ellipsoidal distance, from the centroid of the protein, with the hydrophobicity of the smoothed and inverse transform of the prominent amplitudes for each amino acid residue along the sequence of the B chain of the immunoglobulin C1 set domain protein, 1CD1 according to an embodiment of the present invention;

FIG. 19 is a ribbon diagram illustrating the amino acid residues in each sheet of the B chain of the protein 1CD1 that are nearest the centroid of the structure;

FIG. 20 is a collection of spectral graphs illustrating percent Fourier power amplitudes of the sequence distance, exposure and hydrophobicity over the range of wavelengths from two to four amino acid residues, for the protein 1BMG according to an embodiment of the present invention;

FIG. 21 is a collection of spectral graphs illustrating percent Fourier power amplitudes of the sequences of distance, exposure and hydrophobicity as a function of wavelength for the cuprodoxin protein 1QHQ according to an embodiment of the present invention;

FIG. 22 is a ribbon diagram representation of the cuprodoxin protein 1QHQ;

FIG. 23 is a spectral graph illustrating the values of ellipsoidal distance, from the centroid of the protein, with the hydrophobicity of the smoothed and inverse transform of the prominent amplitudes for each amino acid residue along the sequence of the protein 1QHQ according to an embodiment of the present invention;

FIG. 24 is a collection of spectral graphs illustrating percent Fourier power amplitudes of the sequences of distance, exposure and hydrophobicity, as a function of wavelength, for the protein 1AAC according to an embodiment of the present invention;

FIG. 25 is a spectral graph illustrating the values of ellipsoidal distance, from the centroid of the protein, with the hydrophobicity of the smoothed and inverse transform of the prominent amplitudes for each amino acid residue along the sequence of the protein 1AAC according to an embodiment of the present invention;

FIG. 26 is a collection of spectral graphs illustrating percent Fourier power amplitudes of the sequences of distance, exposure and hydrophobicity as a function of wavelength, for the A chain of protein 1F56 according to an embodiment of the present invention;

FIG. 27 is a spectral graph illustrating the values of ellipsoidal distance, from the centroid of the protein, with the hydrophobicity of the smoothed and inverse transform of the prominent amplitudes for each amino acid residue along the sequence of the A chain of the protein 1F56 according to an embodiment of the present invention;

FIG. 28 is a collection of spectral graphs illustrating percent Fourier amplitudes of the sequences of distance, exposure and hydrophobicity over the range of wavelengths from two to three amino acid residues, for the A chain of the protein 1F56 according to an embodiment of the present invention;

FIG. 29 is a chart illustrating the correlation coefficients of cysteine proteinase papain-like domains according to an embodiment of the present invention;

FIG. 30 is a collection of spectral graphs illustrating percent Fourier power amplitudes of the sequences of distance, exposure and hydrophobicity as a function of wavelength, for the protein domain 1CV8 according to an embodiment of the present invention;

FIG. 31 is a collection of spectral graphs illustrating percent Fourier power amplitudes of the sequences of distance, exposure and hydrophobicity as a function of wavelength, for the A chain of the protein 1ICF according to an embodiment of the present invention;

FIG. 32 is a spectral graph illustrating the values of ellipsoidal distance, from the centroid of the protein, along with the hydrophobicity of the smoothed and the inverse transform of the prominent amplitudes for each amino acid residue along the sequence of the protein 1CV8 according to an embodiment of the present invention;

FIG. 33 is a spectral graph illustrating the values of ellipsoidal distance, from the centroid of the protein with the hydrophobicity of the smoothed and inverse transform of the prominent amplitudes for each amino acid residue along the sequence of the A chain of the protein 1ICF according to an embodiment of the present invention;

FIG. 34 is a ribbon diagram illustrating the location of the three most interior amino acid residues, TYR27, ILE103 and MET122, of the nine-fold prominent period of the protein 1CV8;

FIG. 35 is a collection of spectral graphs illustrating percent Fourier power amplitudes of the sequences of distance, exposure and hydrophobicity as a function of wavelength, for the protein 1GEC according to an embodiment of the present invention;

FIG. 36 is a collection of spectral graphs illustrating percent Fourier power amplitudes of the sequences of distance, exposure and hydrophobicity as a function of wavelength, for the protein 1YAL according to an embodiment of the present invention;

FIG. 37 is a spectral graph illustrating the values of ellipsoidal distance, from the centroid of the protein, along with the hydrophobicity of the smoothed and inverse transform of the prominent amplitudes for each amino acid residue along the sequence of the protein 1GEC according to an embodiment of the present invention; and

FIG. 38 is a spectral graph illustrating the values of ellipsoidal distance, from the centroid of the protein, with the hydrophobicity of the smoothed and inverse transform of the prominent amplitudes for each amino acid residue along the sequence of the protein 1YAL according to an embodiment of the present invention.

### DETAILED DESCRIPTION OF PREFERRED EMBODIMENTS

FIG. 1 is a diagram illustrating an exemplary methodology for characterizing a protein structure. In step 102 of FIG. 1, a protein structure comprising a plurality of amino acid residues is provided for characterization. The present methodologies are directed to the characterization of complete protein structures, or alternatively, portions thereof. Further, the protein structures provided may comprise native protein structures, engineered protein structures, e.g., those engineered to resemble native protein structures, or both native and engineered protein structures.

In step 104 of FIG. 1, distance values representing the distance from the center of the protein to one or more of the amino acid residues making up the protein are determined. As will be described in detail below, the center of the protein structure may comprise the centroid of the protein structure. The term “centroid”’ as used herein, is intended to represent the center of geometry of a given structure. Thus, for example, the centroid of the protein structure denotes the center of geometry of the protein structure. Further, the centroid may or may not correlate with the center of mass. Therefore, centroid, as used herein, should not be considered to be synonymous with the center of mass. The centroid of the protein structure, as will be described in detail below, may be determined based on the positioning of the amino acid residues making up the protein structure. For example, each amino acid residue has a centroid and the centroid of the protein structure can be determined, e.g., as the centroid of the amino acid residue centroids. As will also be further described in detail below, the distance values may be determined using an ellipsoidal distance metric.

In step 106 of FIG. 1, hydrophobicity values, e.g., for each of the amino acid residues described in step 104, above, are obtained, e.g., from the Neumaier hydrophobicity scale, as will be described in detail below. The hydrophobicity values of the amino acid residues describe the overall hydrophobic character of the protein structure. Further, while the present techniques are described in the context of using the Neumaier hydrophobicity scale, it is to be understood that any amino acid hydrophobicity scale may be employed.

In step 108 of FIG. 1, solvent exposure values, e.g., for each of the amino acid residues described in steps 104 and 106, above, are obtained. As described above in step 106, the solvent exposure values may be obtained from a known source of these values. Similar to the hydrophobicity values, as described above, the solvent exposure values of the amino acid residues describe the overall hydrophobic character of the protein structure.

In step 110 of FIG. 1, the distance values, the hydrophobicity values and the solvent exposure values are compared. As will be described in detail below, Fourier transforms are performed on each of the distance values, the hydrophobicity values and the solvent exposure values to aid in the comparison. Further, as will be described in detail below, the hydrophobicity values and the solvent exposure values may be further processed to aid in the comparison.

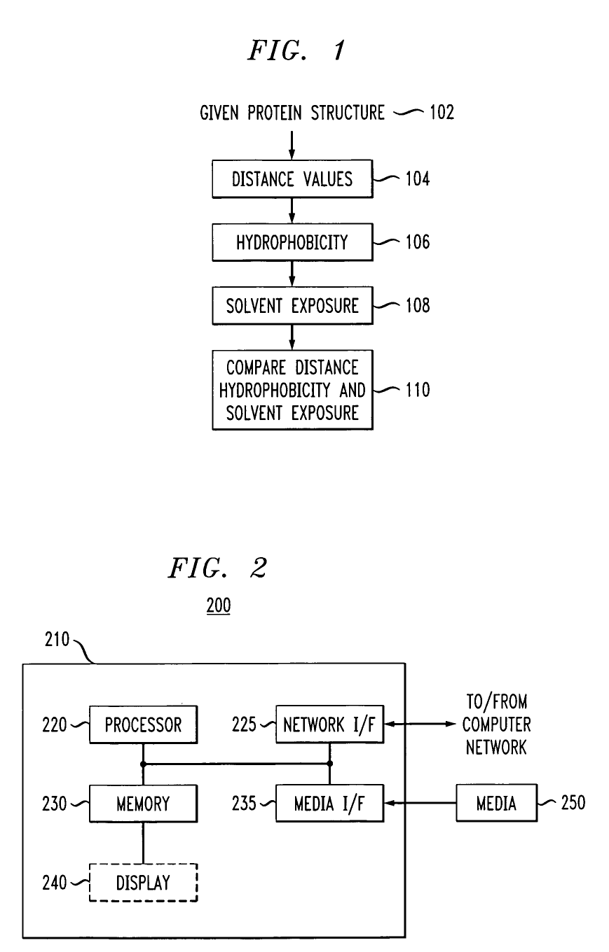

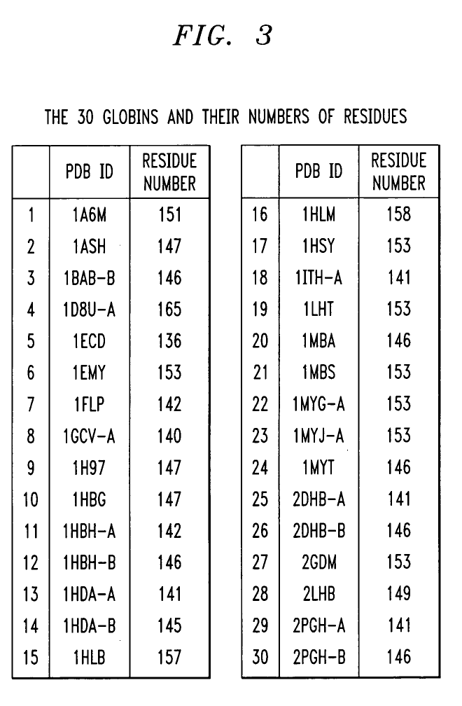

FIG. 2 is a diagram illustrating an exemplary system for characterizing a protein structure. Apparatus 200 comprises a computer system 210 that interacts with media 250. Computer system 210 comprises a processor 220, a network interface 225, a memory 230, a media interface 235 and an optional display 240. Network interface 225 allows computer system 210 to connect to a network, while media interface 235 allows computer system 210 to interact with media 250, such as a Digital Versatile Disk (DVD) or a hard drive.

As is known in the art, the methods and apparatus discussed herein may be distributed as an article of manufacture that itself comprises a computer-readable medium having computer-readable code means embodied thereon. The computer-readable program code means is operable, in conjunction with a computer system such as computer system 210, to carry out all or some of the steps to perform one or more of the methods or create the apparatus discussed herein. For example, the computer-readable code is configured to implement a method of characterizing at least a portion of a protein structure comprising amino acid residues, by the steps of: determining a set of values characterizing the protein structure, wherein each value represents a distance from a center of the protein structure to a center of a given one or more of the amino acid residues; obtaining one or more other sets of values characterizing the hydrophobicity of the protein structure; performing a Fourier transform on each of the sets of values to obtain transformed value sets; and comparing the transformed value sets to correlate the hydrophobicity with the protein structure. The computer-readable medium may be a recordable medium (e.g., floppy disks, hard drive, optical disks such as a DVD, or memory cards) or may be a transmission medium (e.g., a network comprising fiber-optics, the world-wide web, cables, or a wireless channel using time-division multiple access, code-division multiple access, or other radio-frequency channel). Any medium known or developed that can store information suitable for use with a computer system may be used. The computer-readable code means is any mechanism for allowing a computer to read instructions and data, such as magnetic variations on a magnetic medium or height variations on the surface of a compact disk.

Memory 230 configures the processor 220 to implement the methods, steps, and functions disclosed herein. The memory 230 could be distributed or local and the processor 220 could be distributed or singular. The memory 230 could be implemented as an electrical, magnetic or optical memory, or any combination of these or other types of storage devices. Moreover, the term “memory” should be construed broadly enough to encompass any information able to be read from or written to an address in the addressable space accessed by processor 220. With this definition, information on a network, accessible through network interface 225, is still within memory 230 because the processor 220 can retrieve the information from the network. It should be noted that each distributed processor that makes up processor 220 generally contains its own addressable memory space. It should also be noted that some or all of computer system 210 can be incorporated into an application-specific or general-use integrated circuit.

Optional video display 240 is any type of video display suitable for interacting with a human user of apparatus 200. Generally, video display 240 is a computer monitor or other similar video display.

Early recognition of the relationship between amino acid residue hydrophobicity and three-dimensional protein structure was made in W. Kauzmann, Some Factors in the Interpretation of Protein Denaturation, 14 ADV. PROTEIN CHEM. 1-63 (1959), the disclosure of which is incorporated by reference herein. See also, A. M. Lesk, Hydrophobicity—Getting Into Hot Water, 105 BIOPHYS. CHEM. 179-182 (2003), the disclosure of which is incorporated by reference herein. This observation was substantiated subsequently by x-ray studies of haemoglobin and then later by calculations on a number of three-dimensional soluble protein structures. See, M. F. Perutz et al., Structure and function of Haemoglobin, 13 J. MOL. BIOL. 669-678 (1965), G. D. Rose, Prediction of Chain Turns in Globular Soluble Proteins on a Hydrophobic Basis, 272 NATURE 586-590 (1978), H. Meirovitch et al., Empirical Studies of Hydrophobicity. 3. Radial Distribution of Clusters of Hydrophobic and Hydrophilic Amino Acids, 14 MACROMOLECULES 340-345 (1981) and J. Kytte et al., A Simple Method for Displaying the Hydrophobic Character of a Protein, 157 J. MOL. BIOL. 105-132 (1982) (hereinafter “Kytte”), the disclosures of which are incorporated by reference herein.

While these works provided a basis for the concept of the “hydrophobic core” of globular soluble proteins, local regions along the protein chain that are hydrophilic, on average, were also shown to correlate with proximity to the protein exterior. See for example, G. D. Rose et al., Hydrophobic Basis of Packing in Globular Proteins, 44 PROC. NATL. ACAD. SCI. 4643-47 (1980) (hereinafter “Rose 1980”), Kytte and A. Kidera, Relation Between Sequence Similarity and Structural Similarity in Proteins. Role of Important Properties of Amino Acids, 4 J. PROTEIN CHEM. 265-297 (1985), the disclosures of which are incorporated by reference herein. More recent studies have related the sequence of amino acid residue hydrophobicity to the periodicities of protein secondary structures, as well as to the patterns, repeats, periodicities or folds of protein tertiary structures. See for example, Y. Huang et al., Nonlinear Deterministic Structures and the Randomness of Protein Sequences, 17 CHAOS, SOLITONS AND FRACTALS 895-900 (2003) (hereinafter “Huang”), A. J. Mandell et al., Wavelet Transformation of Protein Hydrophobicity Sequences Suggests Their Memberships in Structural Families, A 244 PHYSICA 254-262 (1997) (hereinafter “Mandell”), S. Rackovsky “Hidden” Sequence Periodicities and Protein Architecture, 95 PROC. NATL. ACAD. SCI. 8580-84 (1998), K. B. Murray, Wavelet Transforms for the Characterization and Detection of Repeating Motifs, 316 J. MOL. BIOL. 341-363 (2002) (hereinafter “Murray”), the disclosures of which are incorporated by reference herein.

Concurrent with these developments a number of other studies have shown the sequence of amino acid residue hydrophobicity to be either random, e.g., S. H. White et al., Statistical Distribution of Hydrophobic Residues Along the Length of Protein Chains, 57 BIOPOHYSICAL JOURNAL 911-921 (1990) and Huang, the disclosures of which are incorporated by reference herein, to approximate random sequences slightly edited, e.g., O. B. Ptitsyn, Protein Structures and Neutral Theory of Evolution, 4 J. BIOMOL. STRUCT. DYNAMICS 137-156 (1986) and O. Weiss et al., Information Content of Protein Sequences, 206 J. THEOR. BIOL. 379-386 (2000), the disclosures of which are incorporated by reference herein, or to exhibit systematic deviations superposed upon a random background, e.g., V. J. Pande et al., Nonrandomness in Protein Sequences: Evidence for a Physically Driven Stage of Evolution, 91 PROC. NATL. ACAD. SCI. 12972-75 (1994), A. Irback et al., Evidence for Nonrandom Hydrophobicity Structures in Protein Chains, 93 PROC. NATL. ACAD. SCI. 9533-38 (1996), R. Swartz et al, Frequencies of Amino Acid Strings in Globular Soluble Protein Sequences Indicate Suppression of Blocks of Conservative Hydrophobic Residues, 10 PROTEIN SCIENCE 1023-31 (2001) and Z. A. Yu et al, Multifractal and Correlation Analyses of Protein Sequences From Complete Genomes, 68 PHYS. REV. E. 021913-1-021913-10 (2003), the disclosures of which are incorporated by reference herein.

While patterns have been observed that have been associated with secondary structural features, e.g., S. Vasquez et al., Favored and Suppressed Patterns of Hydrophobic and Nonhydrophobic Amino Acids in Protein Sequences, 90 PROC. NATL. ACAD. SCI. 9100-104 (1993), S. H. White et al., Statistical Distribution of Hydrophobic Residues Along the Length of Protein Chains, 36 J. MOL. EVOL. 76-95 (1993), A. Irback et al., Evidence for Nonrandom Hydrophobicity Structures in Protein Chains, 93 PROC. NATL. ACAD. SCI. 9533-38 (1996) and O. Weiss et al., Measuring Correlations in Protein Sequences, 204 ZEITSCHRIFT FUR PHYSIKALISCHE CHEMIE 183-197 (1998), the disclosures of which are incorporated by reference herein, none of the observed differences from a random background, obtained from a diverse universe of protein sequences, have been attributed to any aspect of protein tertiary structure. Consequently, a question that arises is, how might patterns in the sequence of amino acid residue hydrophobicity correlate with, or be associated with, the patterns, repeats, periodicities or folds of protein tertiary structure within the context of a hydrophobicity distribution that appears to be predominantly random. Furthermore, to what degree or extent do details at the sequence level of amino acid residue hydrophobicity correlate with the proximity of amino acids from the protein interior and in effect with a key feature of protein tertiary structure?

Analysis utilizing the present techniques was performed on thirty globin proteins, nine domains of the immunoglobulin C1 set family, nine domains of the cuprodoxin plastocyanin/azurin family and nine cysteine proteinase papain-like domains. The domain classification was provided using the structural classification of proteins (SCOP) database, described, for example, in A. G. Murzin et al., SCOP: A Structural Classification of Proteins Database for the Investigation of Sequences and Structures, J. MOL. BIOL. 536-540 (1995), the disclosure of which is incorporated by reference herein.

Several notable objectives determined this choice of protein test set, including, to enable comparison between structures composed predominantly of a single type of secondary structure, to enable comparison between structures with the same fold and close in amino acid residue identity, to enable comparison between structures of the same fold but distant in amino acid residue identity, to investigate structures composed of mixed secondary structural types.

In an exemplary embodiment of the present invention, as will be described in detail below, discrete Fourier transforms were obtained for values of the sequences of amino acid residues making up the test set proteins. The values included amino acid residue ellipsoidal distance, solvent exposure and hydrophobicity.

The discrete Fourier transform of the sequence of amino acid residue ellipsoidal distance enables explicit selection of the hydrophobic periodicities that correlate with the periodic excursions of the amino acid residues from the interior-to-exterior of protein structures.

Determining amino acid residue ellipsoidal distance, e.g., using the centroid of amino acid residue centroids, is described in B. D. Silverman, Hydrophobic Moments of Tertiary Protein Structures, 53 PROTEINS: STRUCT. FUNCT. GENET. 880-88 (2003) (hereinafter “Silverman”) and in U.S. patent application Ser. 10/616,880, entitled Moment Analysis of Tertiary Protein Structures, the disclosures of which are incorporated by reference herein. In Silverman, determining the amino acid residue ellipsoidal distance is based upon the amino acid residue locations of the protein. The center-of-geometry of the ith residue, or residue centroid \vec{r}ᵢ is calculated with inclusion of only the backbone α-carbon atom and exclusion of the hydrogen atoms. This distribution of points in three-dimensional space enables calculation of the geometric center \vec{r}_{c}, namely, the centroid of the amino acid residue centroids:

$${{r}^{->}}_{c} = \frac{1}{n} {\sum }_{i} {{r}^{->}}_{i} , \qquad \{ 1 \}$$

wherein n is the total number of amino acid residues.

Linear hydrophobic imbalance about the average value of protein hydrophobicity \overline{h}, is given by the following first-order hydrophobic moment:

$${{h}^{->}}_{1} = \frac{1}{n} {\sum }_{i} ( {h}_{i} - \overline{h} ) {{r}^{->}}_{i} , \qquad \{ 2 \}$$

wherein \vec{h}₁ is invariant with respect to the choice of the origin of the moment expansion since the subtraction of the mean of the distribution yields a distribution, (hᵢ−\overline{h}), with vanishing zero-order moment. The origin of the distribution, hᵢ, that yields the value of \vec{h}ᵢ in Equation 2, is the amino acid residue centroids, \vec{r}_{c}. Namely,

$$\overline{h} = \frac{1}{n} {\sum }_{i} {h}_{i} ,$$

enables Equation 2 to be written as:

$${{h}^{->}}_{1} = \frac{1}{n} {\sum }_{i} {h}_{i} ( {{r}^{->}}_{i} - {{r}^{->}}_{c} ) . \qquad \{ 3 \}$$

The first-order hydrophobic imbalance about the mean value of hydrophobicity is therefore given by a global linear hydrophobic moment calculated with the centroid of the amino acid residue centroids as origin. Thus, the centroid of amino acid residue centroids is used as a spatial origin of the global linear hydrophobic moment. Identification of the spatial origin of the global linear hydrophobic moment expansion enables explicit registration of the global linear hydrophobic moment with the underlying tertiary protein structure.

An ellipsoidal characterization of protein shape is obtained by defining a second rank geometric tensor as follows:

$$\tilde{G} = {\sum }_{i} ( \tilde{1} {\vert {{r}^{->}}_{i} - {{r}^{->}}_{c} \vert}^{2} - ( {{r}^{->}}_{i} - {{r}^{->}}_{c} ) ( {{r}^{->}}_{i} - {{r}^{->}}_{c} ) ) , \qquad \{ 4 \}$$

wherein \tilde{1}, the unit dyadic. The second rank tensor is diagonalized to provide the moments-of-geometry, g₁, g₂ and g₃. These moments-of-geometry are the moments-of-inertia of a discrete distribution of points of unit mass. The moments-of-geometry are linearly related to the moments described in M. H. Hao et al., Effects of Compact Volume and Chain Stiffness on the Conformations of Native Proteins, 89 PROC. NATL. ACAD. SCI. 6614-18 (1992), the disclosure of which is incorporated by reference herein, obtained by writing the geometric tensor in a more symmetric form.

The aspect ratios of the moments-of-geometry provide an ellipsoidal characterization of protein shape:
g₁xₚ²+g₂yₚ²+g₃zₚ²=d², {5}
wherein xₚ, yₚ, zₚ, are coordinates in the frame of the principal axes with the centroid of the protein structure as origin. If the magnitudes are ordered as:
g₁<g₂<g₃, {6}
then the major principal axis is of extent, equal to the square root of d²/g₁, wherein each ith amino acid residue at location xᵢₚ, yᵢₚ, zᵢₚ, in the principal axis frame, can be considered to reside on an ellipsoid with major principal axis equal to the square root of dᵢ²/g₁, namely:
g₁xᵢₚ²+g₂yᵢₚ²+g₃zᵢₚ²=dᵢ². {7}

For a compact protein, the amino acid residue with the largest dᵢ can specify the ellipsoid defining a presumed protein surface. Amino acid residues with the same dᵢ, namely, amino acid residues residing on the same ellipsoid are at the same radial fractional distance from the protein centroid to the protein ellipsoidal surface. Rewriting Equation 7 as:
xᵢₚ²+g′₂yᵢₚ²+g′₃zᵢₚ²=d′ᵢ², {8}
with g′₂=g₂/g₁; g′₃=g₃/g₁; d′²=dᵢ²/g₁ {9}
enables d′ᵢ to be used as the measure of the radial fractional distance of the ith amino acid residue from the center of the protein to the protein surface.

The correlation between amino acid residue distance and amino acid residue solvent accessibility is enhanced with use of this ellipsoidal metric. Thus, when defining the global linear hydrophobic moment, each amino acid residue centroid contributes a magnitude and direction to the global linear hydrophobic moment. Further, each amino acid residue centroid having the same fractional distance to the surface of the tertiary protein structure will contribute an equivalent magnitude to the global linear hydrophobic moment for amino acid residues of equivalent hydrophobicity.

Therefore, use of the term “ellipsoidal” refers to the fact that the correction is made to correlate the distance values more closely with solvent exposure. The distance d is just the value of the principal major axis of the nested ellipsoid upon which the amino acid residue centroid is found. This ellipsoid is nested within a more global ellipsoid characterizing the overall protein shape.

Solvent accessible surface area for each of the amino acid residues may be obtained from the Sealy Center for Structural Biology, University of Texas Medical Branch, Galveston Tex. The Neumaier amino acid residue hydrophobicity scale may be used for amino acid residue hydrophobicity values. This scale, obtained by a principal component analysis of forty-seven different scales, when used in accordance with the present techniques yielded optimal correlation between protein ellipsoidal distance and solvent accessibility with hydrophobicity.

As mentioned above, the present techniques utilize values of the power amplitudes of the frequencies of the discrete Fourier transform of amino acid residue ellipsoidal distance, a new distance metric, to enable explicit identification of frequencies in the hydrophobicity spectrum that correlate with spectral features of tertiary protein structure. Analysis is performed on a number of globins, immunoglobulins, cuprodoxins and papain-like structures. The power amplitude of the frequencies identified and extracted from the hydrophobicity spectrum is shown to be a fraction of the total spectral power and consequently one reason that a straightforward statistical analysis of a universe of protein sequences might miss these long-range correlations. Further, as will be described in detail below, the inverse transforms of the extracted frequencies will be shown to underlie the smoothed hydropathy plots often used to highlight protein three-dimensional structural features. The inverse transforms of the extracted periodicities also quantitatively correlate with the distances.

The discrete Fourier transform provides power amplitudes for a set of N/2 wavelengths, with N being the total number of amino acid residues. The number of wavelengths that span the sequence of amino acid residues is a convenient measure of frequency and will be used throughout the present description. The number of amino acid residues spanned will designate the wavelength. For example, a frequency of one is associated with a single wavelength that spans the entire sequence, while a frequency of N/2 is associated with a wavelength that spans two amino acid residues.

Consequently, with regard to the amino acid residue interval for proteins of one hundred or more amino acid residues, the resolution is sparse at long wavelengths and fine at short wavelengths. At long wavelengths, the resolution, therefore, presents no problem when comparing the power amplitudes of two different sequences. At short wavelengths, the fine resolution of the individual wavelengths requires comparison of power amplitudes over finite spectral ranges. The amplitude associated with the largest wavelength of frequency one, the single wavelength that spans the entire sequence, selects that part of the pattern of the amino acid sequence that is non-repeating. This action would signify the average hydrophobic imbalance along the extent of the entire protein chain.

Significant power amplitudes at frequencies of the distance spectrum highlight oscillations that characterize major structural motifs of the protein. These structural motifs are conspicuous motifs that represent the periodic transit of the amino acid residues of the protein chain from the interior-to-exterior environments.

In addition to major structural motifs, the present techniques are also used to identify sets of frequencies of prominent power amplitudes, namely those sets having a power amplitude of the distance value distribution, e.g., spectrum, greater than five percent of the total spectrum. The modes of prominent power amplitude may have substantially comparable amplitudes over several different frequencies, where the prevalence of a single structural periodicity may not be apparent. The identification of frequencies of prominent power amplitude in the distance spectrum enables selection of the modes of the corresponding frequencies of the hydrophobicity spectrum that are shown to correlate with the distances. Consequently, a focus of the present methodology involves identifying modes of prominent power amplitude in the distance spectrum, to enable selection or extraction of the modes of corresponding frequency of the hydrophobicity spectrum.

The power spectra of amino acid residue distances, solvent accessibility, and hydrophobicity may then be visually displayed for each protein sample to enable comparison. For the majority of the power spectra, the modes of prominent power amplitude identified in the distance spectrum are paralleled by pronounced amplitudes at the corresponding frequencies of solvent accessibility and hydrophobicity. The distance and hydrophobicity spectra complement each other. Namely, the distance spectrum has a major power component at long wavelengths and low frequencies whereas the hydrophobicity spectrum has a major power component at short wavelengths and high frequencies. The techniques, therefore, identify features of prominent spectral power enabling selection of those features that are relevant, but of weaker amplitude.

Smoothed hydropathy plots or profiles, are commonly used to characterize protein structural motifs, which has been suggested in connection with wavelet analyses. See, for example, Mandell and Murray. Wavelet analyses, identifying correspondences between values of hydrophobicity and protein structural motifs or repeats, might be viewed as a particular form of “windowing” where a distribution of length scales or window widths is utilized in connection with the mother wavelet, a particular functional form of the window. A particular advantage of the Fourier decomposition is that the complete set of Fourier coefficients allows the amplitudes of the extracted frequencies to be, not only inverted, but also eliminated in inversion from the complete set of amplitudes, enabling investigation of their importance in providing correlation with particular structural features. Thus a number of correlation coefficients can be calculated that provide a quantitative measure of the relative importance of various hydrophobic spectral ranges and individual frequencies in correlating with amino acid distance from the protein interior.

It is shown that the amplitudes correlating with amino acid residue distance from the protein interior generally represent a minor fraction of the total power amplitude of the hydrophobic Fourier spectrum and consequently not inconsistent with a distribution that would appear to be predominantly random. This is consistent with the general observation that sequences with the same fold can have low similarity. Furthermore, the frequencies of these amplitudes and their corresponding wavelengths are distributed over different sets of values that reflect differences in protein folds as well as other structural details.

For globular soluble proteins, sliding window analysis has been used to show that local regions of the smoothed values of amino acid residue hydrophilicity correlated with proximity to the protein exterior. See Rose 1980 and Kytte. Namely, window averaging and smoothing were used to damp out the higher frequency oscillations of the distribution, making the longer wavelength variations of sequence hydrophobicity overt.

Such windowing procedures yielded hydrophobic variations that correlated with the periodic inside environment-to-outside environment structural excursions of tertiary protein structures. However, since the variations are associated with sequence averages, it is of interest to extract the underlying periodicities of the distribution at the amino acid residue level. This is achieved with the present techniques by extracting frequencies in the hydrophobicity spectrum for visual comparison and correlation analyses. As will be described in detail below, the inverse transforms of the extracted periodicities correspond well to the hydrophobic spatial profile obtained by sliding window averaging and smoothing. Interestingly, it is observed that the inverse transform of each individual hydrophobic spectral component has a greater probability of correlating with the distances than not, no matter how low-level. This is a spectral corollary of the hydrophobic core.

The present techniques were performed on globin proteins (all-alpha proteins) and immunoglobulins and cuprodoxins (all-beta proteins) and papain-like structures (mixed alpha/beta).

Globin Proteins—All-Alpha Proteins

In an exemplary embodiment, the present techniques were performed on 30 globin structures. The majority of these structures were selected from the native structures of the hg_structal set used for decoy discrimination. See for example, B. Park et al., Energy Functions that Discriminate X-Ray and Near-Native Folds From Well-Constructed Decoys, 258 J. MOL. BIOL. 367-392 (1996), the disclosure of which is incorporated by reference herein. FIG. 3 is a chart illustrating the 30 protein database (PDB) globin proteins and their corresponding number of amino acid residues.

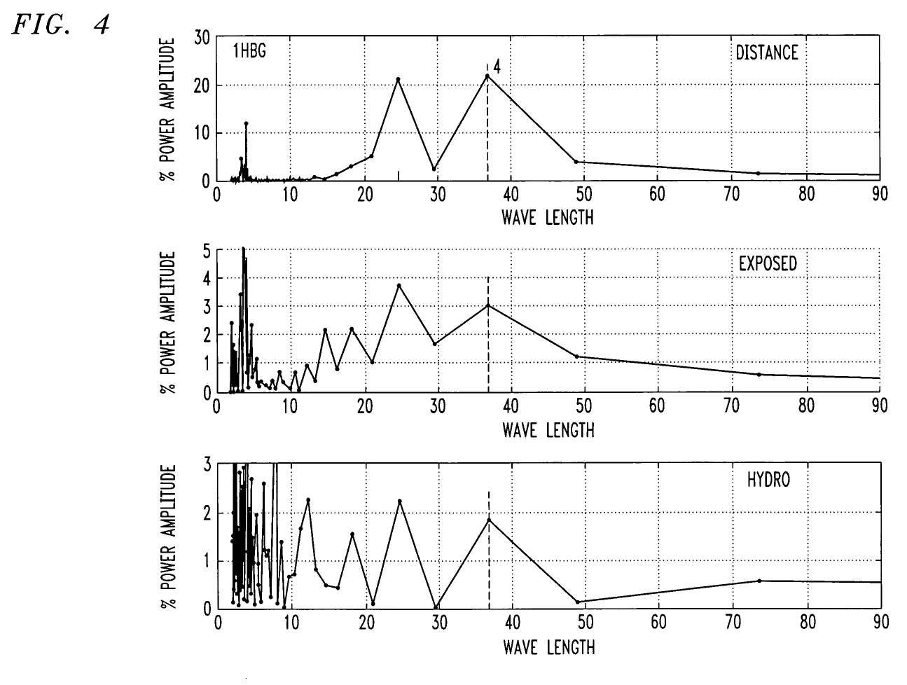

FIG. 4 is a collection of spectral graphs illustrating percent Fourier power amplitudes of the sequences of distance, exposure and hydrophobicity as a function of wavelength, for the protein 1HBG. Namely, the graphs in FIG. 4 illustrate the percentage of the total power at each of the wavelengths of the discrete Fourier transforms of the sequence of amino acid residue ellipsoidal distance, solvent accessibility or exposure, and hydrophobicity for the protein 1HBG (globin structure number 10 in FIG. 3, described above). The abscissa provides the wavelength in units of the numbers of amino acid residues. Frequencies 4, 6, and 7 have been selected since each of these frequencies contain greater than five percent of the total power of the distance spectrum. The correspondence between the relative amplitudes at frequencies 4 and 6 for all three spectra of 1HBG is apparent.

FIG. 4 also shows high frequency components of the distance spectrum with greater than five percent power amplitude in the distance spectrum. These high frequency components associated with the helical secondary structure, are however not selected since the lower frequency spectral components associated with tertiary structure are currently under analysis.

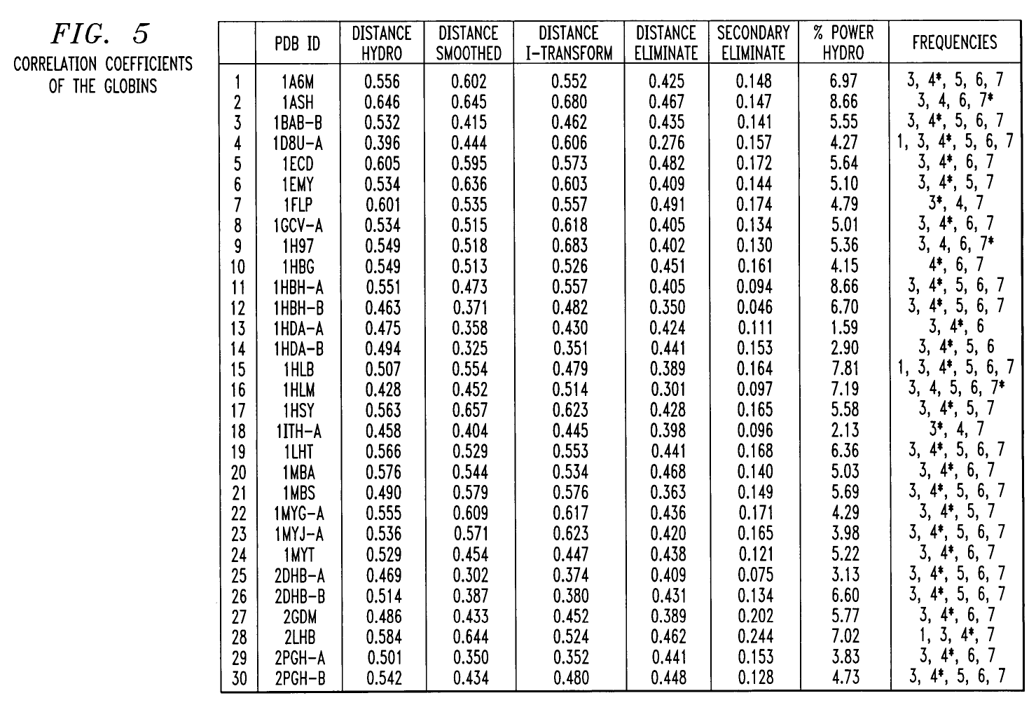

FIG. 5 is a chart illustrating correlation coefficients for the 30 sample globin proteins. Namely, in FIG. 5, the frequencies of prominent power amplitude of the thirty globin proteins are listed (e.g., in the column labeled “frequencies”) along with several correlation coefficients which will be described in detail below. Asterisks are used to indicate frequencies having the greatest power amplitude. These frequencies having the greatest power amplitude are signified by a dashed line in each of the spectral graphs presented in conjunction with the present description.

As mentioned above in conjunction with the description of FIG. 4, the correspondence between the relative amplitudes at frequencies 4 and 6, namely at amino acid residue numbers of 147/4=36.75 and 147/6=24.5, for all three spectra of 1HBG is apparent. This range of wavelengths, greater than 20 amino acid residues, is the range over which all 30 globin proteins exhibit significant power in their ellipsoidal distance spectra. While the percentage of power in the amplitudes of the two frequencies, 4 and 6 of the distance spectrum, is 43 percent of the total power, the percentage of power in the two corresponding frequencies of the hydrophobicity spectrum is only four percent of the total, as is shown listed in the column labeled “% Power hydro” in FIG. 5.

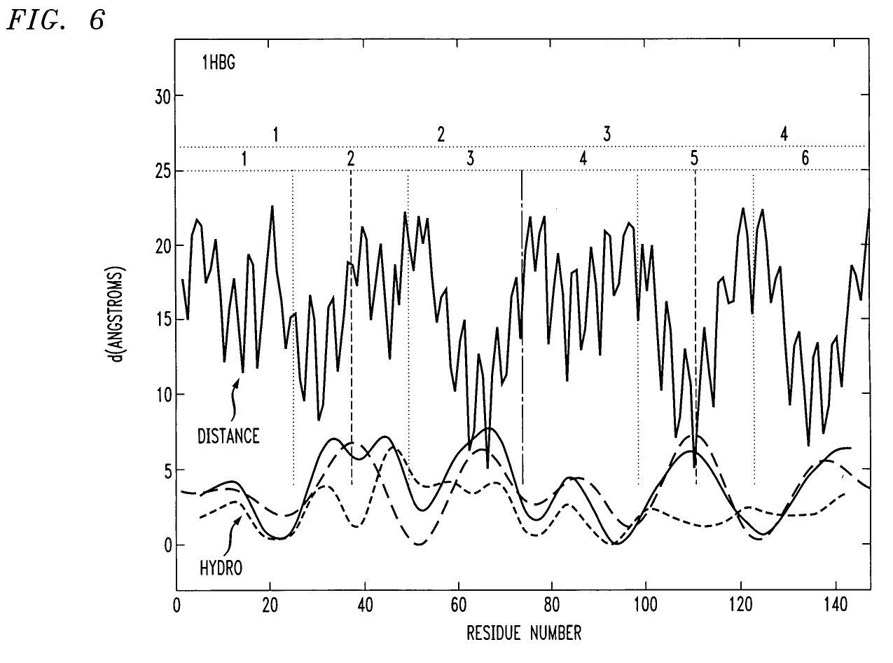

FIG. 6 is a spectral graph illustrating the values of ellipsoidal distance, from the centroid of the protein, with the hydrophobicity of the smoothed (shown as a connected line) and inverse transform of the prominent amplitudes (shown as a dashed line) for each amino acid residue along the sequence of the protein 1HBG. The dotted curve represents the smoothed profile obtained after elimination of the amplitudes at the selected frequencies, e.g., 4, 6 and 7.

The high frequency oscillations in distance mirror the rapid inside environment-to-outside environment excursions of the amino acid residues along the alpha helices. Superposed on the plot are dashed lines dividing the protein chain into the four regions that correspond to the four-fold period observed in the power spectra, as well as dotted lines demarcating the six-fold period.

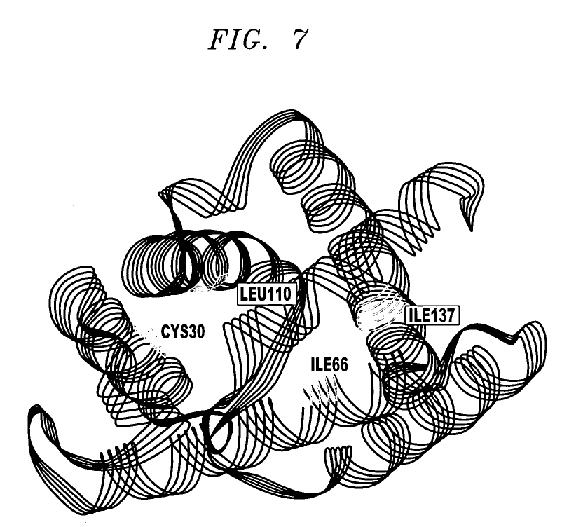

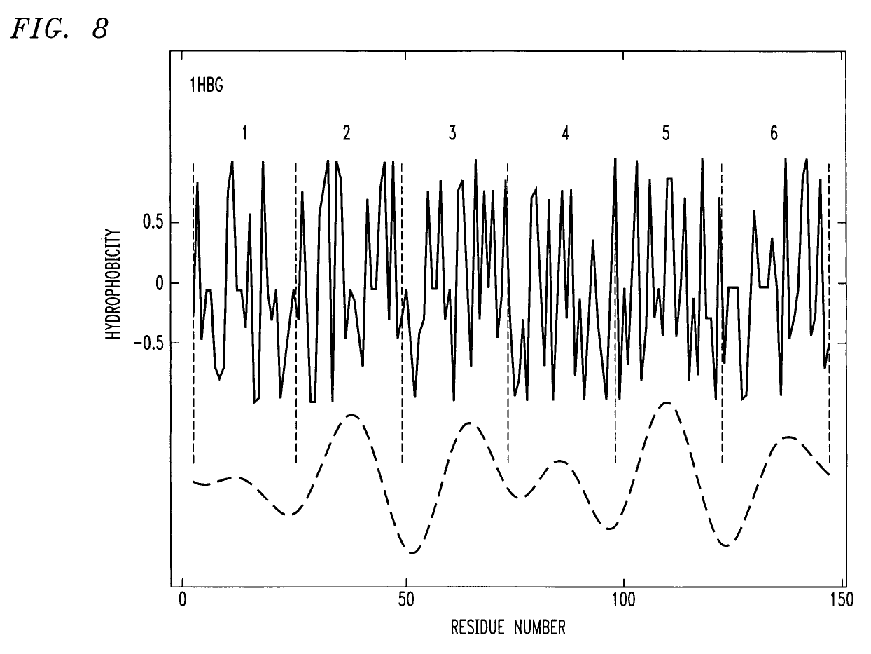

The spatial inside environment-to-outside environment periodicity across the four segments is visually apparent. This periodicity may be referenced to the four minimal distances from the centroid of the protein to certain interior amino acid residues, for example, the amino acid residues CYS30, ILE66, LEU110 and ILE137. Namely, these amino acid residues belong to different helices and the excursion from one to the next is, on average, a distance of roughly one-quarter the length of the chain. The structure of protein 1HBG will be described in detail below, e.g., in conjunction with the description of FIG. 7.

The values of hydrophobicity of the smoothed hydrophobic amplitudes for each amino acid residue along the sequence are shown. Windowing over an extent of eight amino acid residues and splining can be used to damp out the high frequency oscillations associated with secondary structure amphipathicity. Notably, there is a correspondence, or registration, between the valleys of amino acid residue distance from the interior of the protein, and peaks in the locally averaged values of hydrophobicity.

The dashed line, superposed upon the smoothed values, is the inverse transform of the amplitudes of the selected 4, 6 and 7 fold periodicities. The 4 fold and 6 fold periodicities of the composite inverse transform profile are the major periodic components of the smoothed profile. The 4 fold period encompasses the four deepest minima in distance, in the demarcated segments 1 through 4. The 6 fold period encompasses these with the addition of the two minima, less deep, in the six-fold demarcated segments, 1 and 4. Frequency 7 has been included in the inverse transform since its power amplitude at 5.10 just exceeds the arbitrary cut-off power amplitude of five percent.

While most of the smoothed variation is accounted for by just these three frequencies, e.g., 4, 6 and 7, the smoothed values pick up the variations across all seven helices. The inverse transform does not account for the dual peak that registers with the minimal distance of CYS30 that is present within helix 2 and of PHE45 that is present within the irregular helix 3. While the four and six fold periods are easily discernable by examining the profiles of the smoothed and inverse transforms, the visual identification of these periods in the original distribution of the individual values of amino acid hydrophobicity is not as apparent.

FIG. 7 is a ribbon diagram representation of protein 1HBG. Namely, the ribbon diagram in FIG. 7 highlights the location of the four innermost amino acid residues, CYS30, ILE66, LEU110 and ILE137, of protein 1HBG. Such prominent frequency associated with four innermost amino acid residues appears in all the distance spectra of the thirty globin proteins tested.

FIG. 8 is a spectral graph illustrating the inverse transform of the amplitudes of three hydrophobic periodicities, e.g., 4, 6 and 7, superposed upon the original individual amino acid residue values of the hydrophobicity distribution. The protein 1HGB clearly exhibits two distinct correspondences in the distance, exposure and hydrophobicity transforms. Protein structures having the same fold and belonging to the same family, while exhibiting similar overall structural topology, can, however, differ in structural detail. These differences in structural detail can show up as differences in the relative amplitudes of power, at different frequencies of the distance spectrum.

Of the 30 globin proteins examined, the majority have the greatest power amplitude at frequency value 4 (while in fact, the prominent amplitudes range in frequency from between values of 1 to 7, e.g., as may be seen from the “frequencies” column of FIG. 5). There are also differences in the amino acid sequences of each of the globin proteins, and consequently differences in the sequences of amino acid residue hydrophobicity. Despite these differences, however, the majority of the 30 globin proteins exhibit a clear correspondence between the relative amplitudes of the distance and hydrophobicity spectra at the frequency of greatest prominent power amplitude, namely at a frequency of 4. Most of the globin proteins also show relative correspondences at other frequencies of prominent power amplitude.

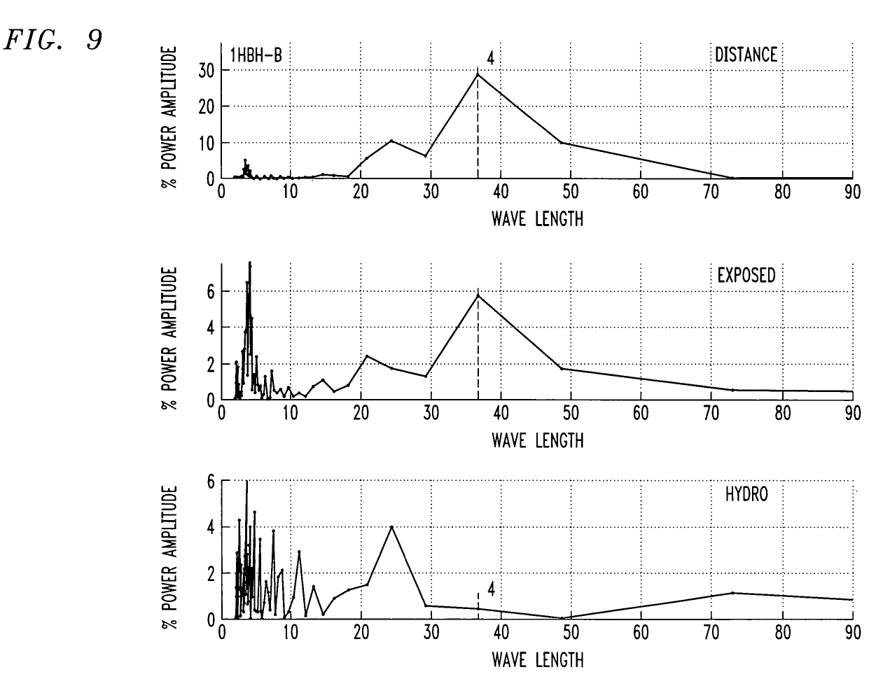

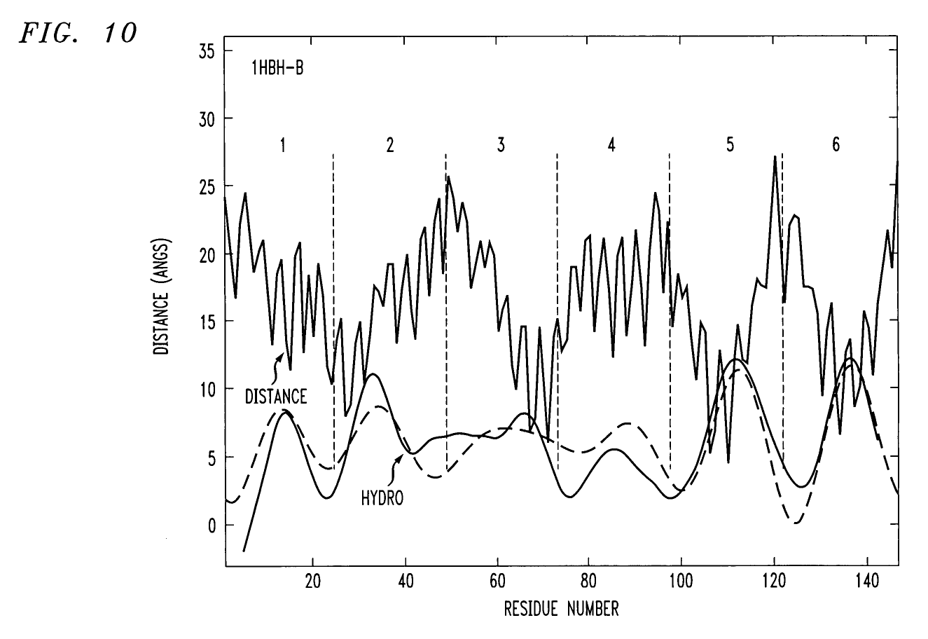

There are, however, exceptions. For three of the 30 globin proteins, namely, 1HBH-B, 1ITH-A and 1MYT, the frequencies of greatest prominent power amplitude in the distance spectrum do not correspond to frequencies of salient power in the hydrophobicity spectrum. FIG. 9 is a collection of spectral graphs illustrating percent Fourier power amplitudes of the sequences of distance, solvent exposure and hydrophobicity as a function of wavelength for the B chain of the protein 1HBH. While the major percentage of power of the distance spectrum is at frequency 4, pronounced power at this frequency is not observed in the hydrophobic spectrum, as had been observed for the protein 1HBG, e.g., as described above in conjunction with the description of FIG. 7.

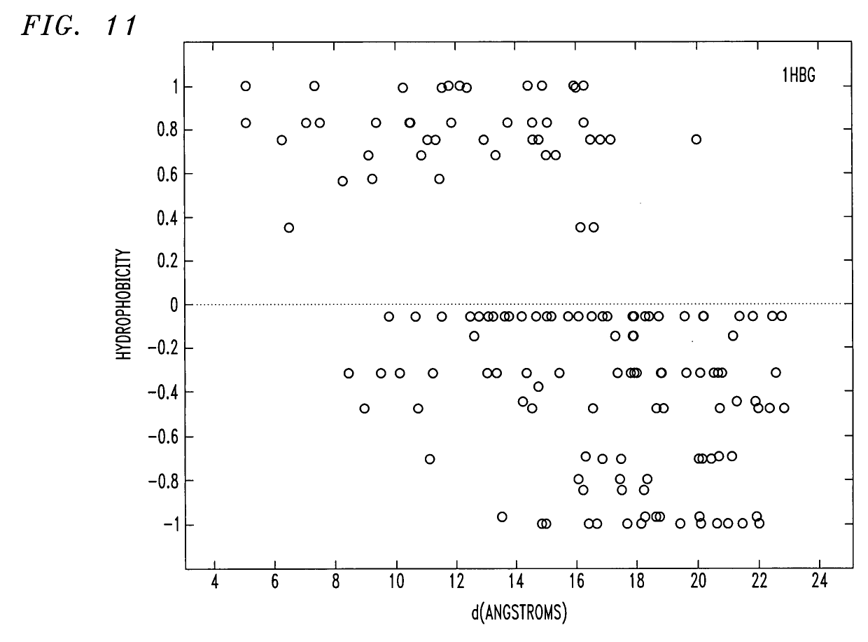

FIG. 10 is a spectral graph illustrating the values of ellipsoidal distance, from the centroid of the protein, with the hydrophobicity of the smoothed (shown as a connected line) and inverse transform of the prominent amplitudes (shown as a dashed line) for each amino acid residue along the sequence of the B chain of the protein 1HBH. Namely, FIG. 10 illustrates a comparison between the distance and hydrophobicity for the protein 1HBH-B

As mentioned, e.g., in conjunction with the description of FIG. 9 above, pronounced power is not observed at frequency 4 in the hydrophobic spectrum. This reduced hydrophobic amplitude at frequency 4 yields less pronounced registration between the maxima in hydrophobicity and minimal value in distance, as is illustrated in segment 3 of FIG. 10. On the other hand, the overall correlation between the values of distance and the values of the inverse transform of the hydrophobic amplitudes is retained.

Calculation of various correlation coefficients can reveal the relative importance of the individual frequencies and spectral ranges in contributing to the correlation between distance and hydrophobicity. FIG. 5, as described above, lists several different correlation coefficients for each of the thirty globin proteins. Namely, the “frequencies” column lists the frequencies of prominent power amplitude extracted for analysis. The “% Power hydro” column lists the percentages of total power of hydrophobicity contributed by the sum of these frequencies. Comparable percentages obtained from the spectral prominent power amplitude of the distances are approximately 50 percent. The column labeled “distance hydro” lists the correlation coefficients between the individual values of amino acid hydrophobicity and distance. For example, this correlation coefficient for the protein, 1HBG, is 0.549. From the perspective of the spectral decomposition, this value includes contributions from the entire spectral range, consisting of the long wavelength oscillations associated with protein tertiary structure, as well as the shorter wavelength oscillations associated with protein amphipathic secondary structure.

With further regard to FIG. 5, described above, the correlation coefficients between the smoothed values of amino acid residue hydrophobicity and distance are listed in the column labeled “distance smoothed.” For example, for the protein 1HBG this value is 0.513. The amplitudes of the selected frequencies are inverted to provide the spatial distribution of hydrophobicity that is correlated with the distances. The values of these correlation coefficients are listed in the column labeled “distance I-transform.” For example, the value for the protein 1HBG is 0.526. Frequencies of prominent power amplitude in the distance spectrum generally select frequencies in the hydrophobicity spectrum that correlate optimally with distance. For example, had the hydrophobic Fourier amplitudes at frequencies 3, 5, and 8 been inverted instead of at 4, 6, and 7, the correlation coefficient with the distances would have been 0.124 instead of 0.526.

With further regard to FIG. 5, described above, it should be noted that the distribution of values in the “distance I-transform” column roughly parallels that obtained by correlating the smoothed values of hydrophobicity with the distance listed in the “distance smoothed” column. These two sets of values, hydrophobicity and distance, provide a quantitative measure that complements a visual comparison of the smoothed and inverse transform values of hydrophobicity with the distances in FIG. 6, described above. It should also be noted that the correlation between the individual values of amino acid residue hydrophobicity and distance, listed in the “distance hydro” column, are moderate and not closely correlative. This is due, not only to the predominant randomness of the sequence of amino acid hydrophobicity, but also to the substantially uniform, but biased, spatial distribution of amino acid residue hydrophobicity from the interior environment.

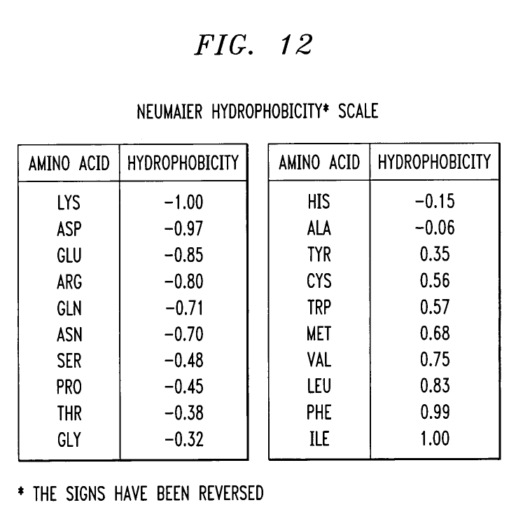

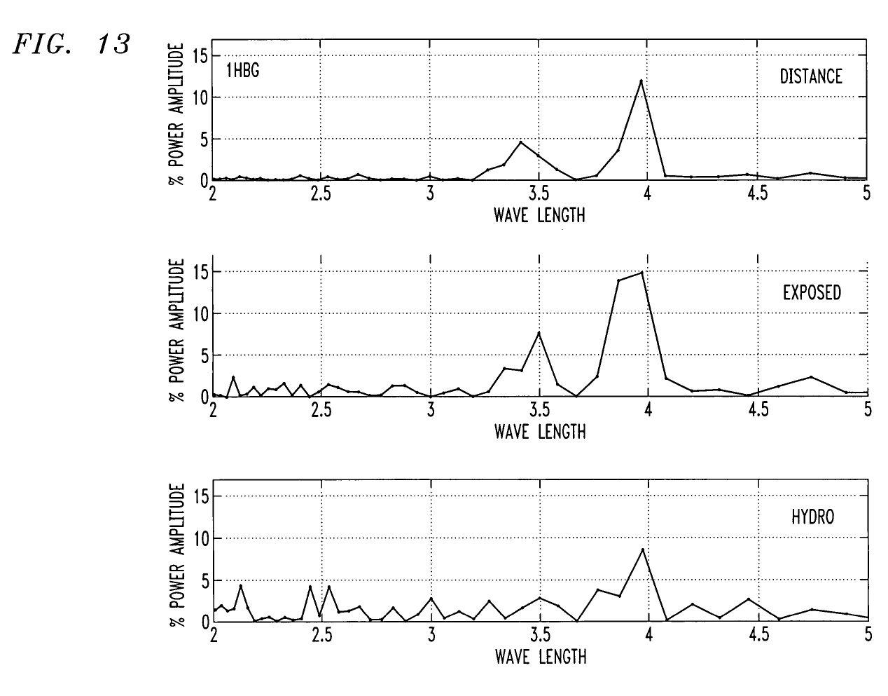

FIG. 11 is a graph illustrating the spatial distribution of amino acid residue hydrophobicity from the interior environment of the protein 1HBG. Namely, FIG. 11 illustrates the individual values of amino acid hydrophobicity at their ellipsoidal distances. FIG. 12 is a chart illustrating Neumaier hydrophobicity scale values for the 20 naturally occurring amino acid residues. In FIG. 12, the signs have been reversed so that the greater the value, the more hydrophobic the amino acid. To this regard, all negative signs of the correlation coefficients listed in the tables have been suppressed.

With further regard to FIG. 5, described above, a quantitative estimate of the importance of the selected frequencies in contributing to the correlation coefficients between the individual values of amino acid residue hydrophobicity and distance, listed in the “distance hydro” column, can be inferred from the values listed in the column labeled “distance eliminate.” These values have been obtained by eliminating Fourier amplitudes of the selected frequencies, from the entire spectral range of frequencies prior to inversion. The correlation coefficient obtained by eliminating the amplitudes of frequencies 4, 6, and 7 of the protein 1HBG is 0.451 (reduced in value from 0.549).

With further regard to FIG. 6, described above, the effect of eliminating the amplitudes of frequencies 4, 6, and 7 upon the smoothed profile of amino acid residue hydrophobicity is shown, for example, by the dotted line in FIG. 6. It is noted that the overall correlation is however affected with correspondences between the distances and smoothed hydrophobicity values most affected in the region near the C terminal end of the chain. Hydrophobicity is seen not to correlate well with distance at this end of the chain. Deleting frequencies that do not exhibit prominent power amplitude highlights the relative unimportance of these deleted frequencies in providing correlation with the distances. For example, the deletion of the amplitudes of the frequencies 3, 5, and 8, for the protein 1HBG, from the full complement of amplitudes, yields a correlation coefficient of 0.537 between the hydrophobic inverse transform and the distances, i.e., namely, a value that differs from the value of 0.549 by only two percent. Moreover, the deletions provide quantitative estimates of the importance of the selected periodicities, as well as of amphipathic spectral ranges of secondary structure, to the correlation between the individual values of residue hydrophobicity and distance.

Further reduction in the correlation coefficient of protein 1HBG is obtained by the further deletion of the high frequency short wavelength range, from two to five amino acid residue wavelengths. This spectral range includes the contributions from amphipathic helical secondary structures. The values of the correlation coefficient after this dual elimination may be observed, for example, in the column labeled “secondary eliminate,” appearing in FIG. 5, described above. For example, the value for the protein 1HBG is 0.161. The significant reduction for all of the values listed in the “secondary eliminate” column is notable. Namely, this reduction in values highlights the relative importance of the amphipathic secondary structure in providing correlation with the interior environment-to-exterior environment excursions of the amino acid residues.

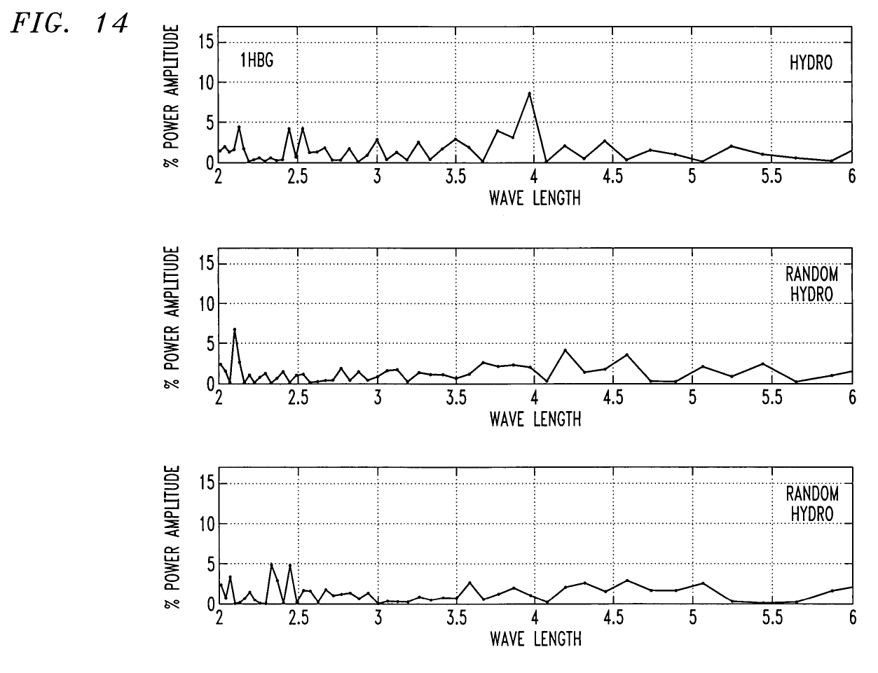

FIG. 13 is a collection of spectral graphs illustrating percent Fourier power amplitudes of the sequences of distance, exposure and hydrophobicity over the range of wavelengths from two to five amino acid residues, for the protein 1HBG. This range encompasses features associated with the secondary structure of the protein 1HBG.

Enhanced power amplitude is seen in the vicinity just below wavelengths of four amino acid residues in all three spectra. For ease of comparison, the ordinate scales have been chosen to be the same in all three frames. The enhanced power amplitude is associated with the helical structure. The hydrophobic power amplitude in this range of wavelengths is, however, not as pronounced as appears in the two other spectra. The power amplitudes of wavelengths that are not in the vicinity of 3.5 to four amino acid residues, namely in the background, are however more pronounced in the hydrophobicity spectrum.

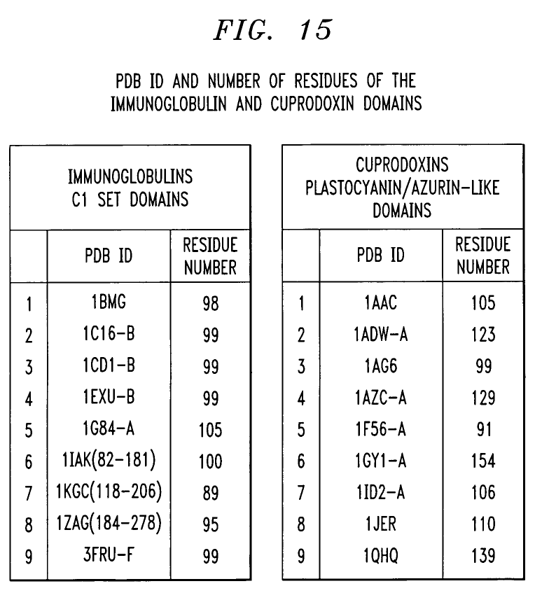

FIG. 14 is a collection of spectral graphs illustrating percent Fourier power amplitudes of the sequence of amino acid residue hydrophobicity over the range of wavelengths from two to five amino acid residues for the protein 1HBG in the top frame, and for two randomized sequences in the lower two frames. Namely, the graphs in FIG. 14 illustrate the hydrophobicity profile of the protein 1HBG verses two randomized sequences for the range of wavelengths from two to five amino acid residues. The hydrophobicity profile of 1HBG is shown in the top frame. The sequence of amino acid residue hydrophobicity is then randomized to obtain the other two frames of data.

The prominent feature below four amino acid residues in wavelength does not appear in either of the randomized distributions. The percentage of the total power, within the range of wavelengths from two to five amino acid residues, shown in the top frame, is 70 percent. Performing thousands of calculations on randomized sequences yields an average percentage of 60 percent of the total power in the same spectral range. Calculations on all 30 globin proteins with multiple randomization runs show this to be a general feature, and that randomization of the sequence of amino acid residue hydrophobicity shifts spectral amplitude out of this range of frequencies. For the globin proteins, at least, this is evidence of a non-random feature of the distribution that reflects the presence of alpha helical amphiphilicity superimposed upon a distribution which appears to be random.

Immunoglobulins and Cuprodoxins—All-Beta Proteins

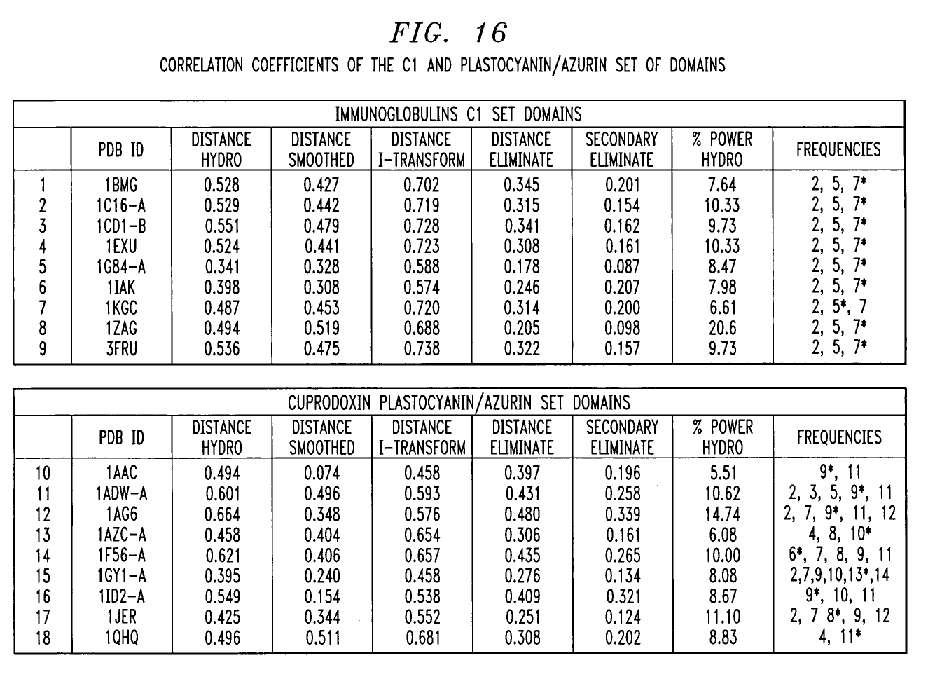

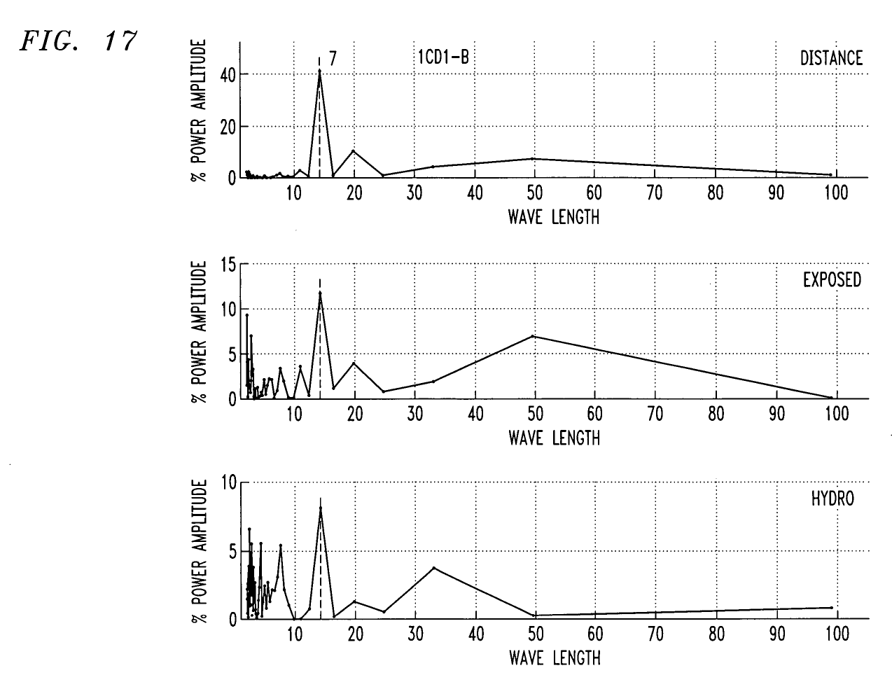

In an exemplary embodiment, the present techniques were performed on 18 all-beta structures, namely nine domains from the family of the immunoglobulin C1 set and nine domains from the cuprodoxin plastocyanin/azurin family. See Murzin et al., SCOP: A Structural Classification of Proteins Database for the Investigation of Sequences and Structures, 247 J. MOL. BIOL. 536-540 (1995), the disclosure of which is incorporated by reference herein. FIG. 15 is a chart illustrating the PDB identification and number of amino acid residues for each of the immunoglobulin and cuprodoxin domains. FIG. 16 is a chart illustrating correlation coefficients of the C1 and plastocyanin/azurin set of domains. Namely, the chart shows the correlation coefficients for each of the immunoglobulin and cuprodoxin domains (which is similar in format to the chart shown in FIG. 5 and described above in conjunction with the description of the globin proteins).

It may be noted in the column labeled “frequencies,” that while the sets of frequencies identified as prominent (e.g., frequencies 2, 5, 7 for 1BMG) are all identical for the immunoglobulins, they vary for the cuprodoxins. The immunoglobulin set of domains is composed primarily of a well-defined set of seven beta sheets. The one exception, the protein 1KGC, has a fewer number of such sheets (resulting in its major component of power amplitude being at a frequency of five instead of seven).

The cuprodoxin set is more structurally heterogeneous, containing a number of short helices or mini-helical regions. Different numbers of amino acid residues of each protein of the cuprodoxin set also contribute to the lack of registration of the frequencies of prominent power amplitude.

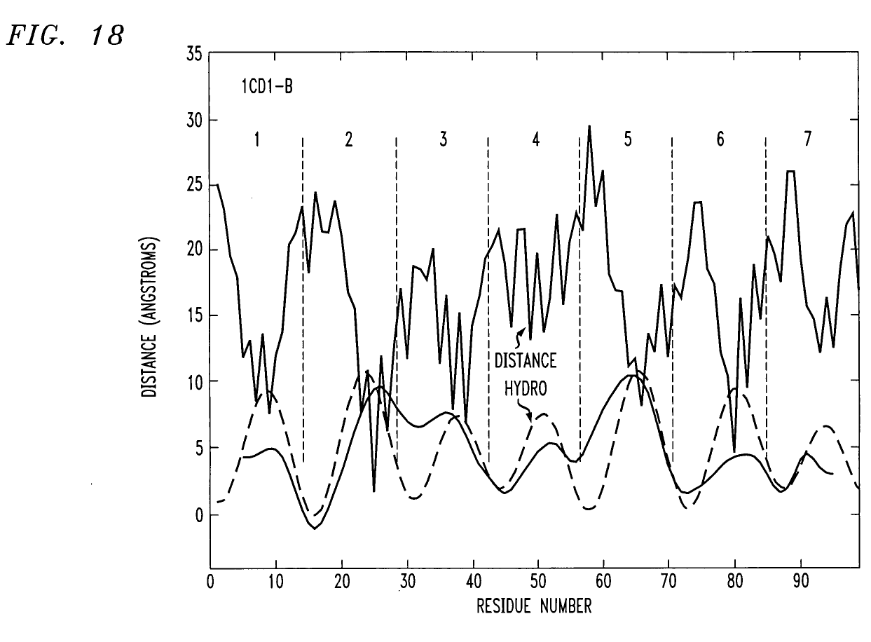

FIG. 17 is a collection of spectral graphs illustrating percent Fourier power amplitudes of the sequences of distance, exposure and hydrophobicity as a function of wavelength, for the B chain of the immunoglobulin C1 set domain protein, 1CD1. Frequencies of prominent power amplitude identified are 2, 5, and 7. Enhanced power amplitude is noticeable at frequency 7 for all three spectra. While the hydrophobic amplitude at period 2 might not appear to contribute to the correlation between distance and hydrophobicity, it is responsible for a three percent enhancement of the correlation coefficient between the individual values of amino acid residue distance and hydrophobicity.

FIG. 18 is a spectral graph illustrating the values of ellipsoidal distance, from the centroid of the protein, with the hydrophobicity of the smoothed (shown as a connected line) and inverse transform of the prominent amplitudes (shown as a dashed line) for each amino acid residue along the sequence of the B chain of the immunoglobulin C1 set domain protein, 1CD1. Namely, the graphs illustrate the amino acid residue distances and hydrophobicity profiles of the B chain of the protein 1CD1. The peaks and valleys of the smoothed hydrophobicity profile and inverse transform of the amplitudes of the prominent frequencies track each other and the peaks register closely with the valleys of the distance profile.

With further regard to FIG. 16, described above, the inverse transform of all nine immunoglobulin structures yields correlation coefficients with distance that are greater in value than those obtained by correlating the smoothed values with distance. The values for the B chain of 1CD1 are 0.728 (see the column labeled “distance I-transform”) and 0.479 (see the column labeled “distance smoothed”), respectively. As was found for the globin proteins, calculations on the C1 set of domains clearly support the strategy of identifying frequencies of prominent power amplitude in the distance spectrum to select those frequencies in hydrophobicity that correlate with distance. For example, had the frequencies 3, 4 and 8 been selected, the inverse transforms of their Fourier amplitudes would yield a correlation coefficient of 0.117 for the protein 1CD1.

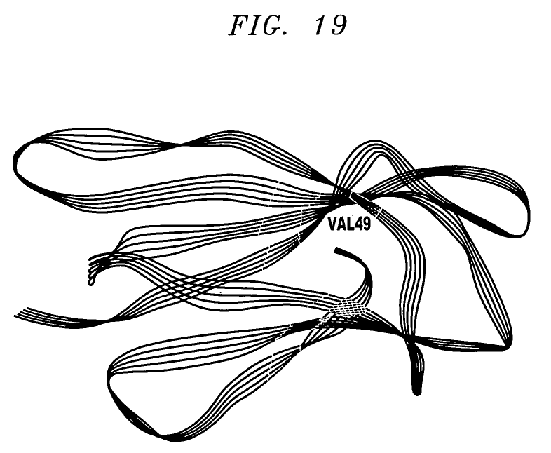

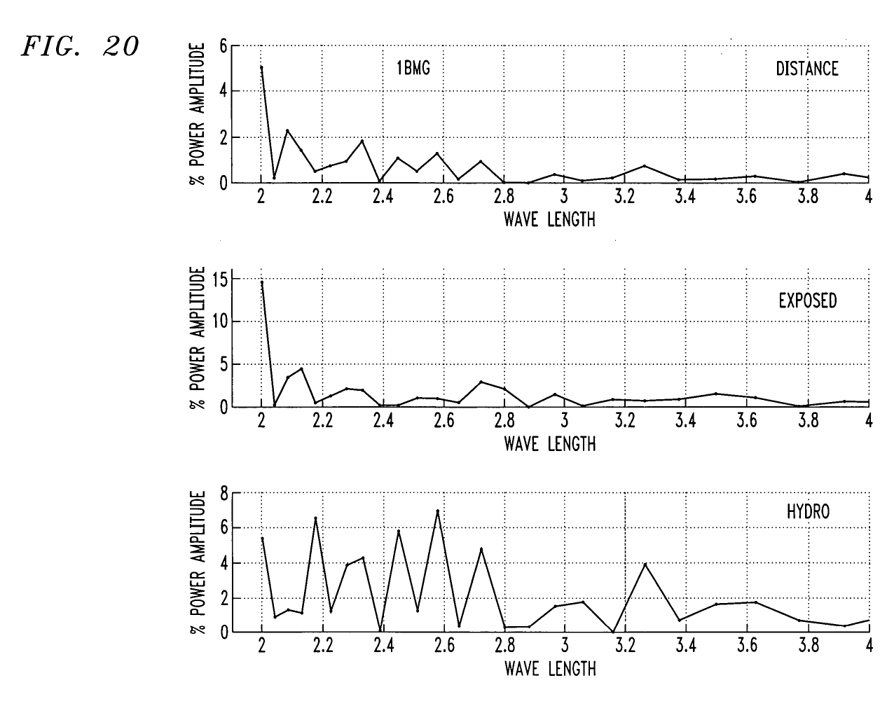

Significant power amplitude at frequency 7 reflects the period of recurrent local minima in distance, as demarcated by the vertical dashed lines in FIG. 18 for the B chain of the protein 1CD1. Amino acid residues at these distances are near the center of each of the beta sheets. The location of these amino acid residues is highlighted in the ribbon diagram of FIG. 19. FIG. 19 is a ribbon diagram illustrating the amino acid residues in each sheet of the B chain of the protein 1CD1 that are nearest the centroid of the structure. In reference to the spectral graphs described above in conjunction with the description of FIG. 17, the peak at frequency 7 can be viewed as a count of the number of beta sheets as each are traversed by moving along the sequence. In FIG. 19, VAL49, while not assigned within a beta sheet in the PDB file, is shown to be in a sheet-like region (e.g., demarcated segment 4 of FIG. 18, described above) near the protein centroid. This sheet-like region is not as close to the centroid of the structure as are the central sheet regions (e.g., as represented by the other demarcated segments of FIG. 18, described above).

Several of the proteins of the C1 set have sequences close in identity, while several are quite different. For example, the protein 1BMG and the B chain of protein 1C16-B have a sequence identity of 77 percent and a root mean square deviation (RMSD) of 1.1 Angstroms after combinatorial extension (CE) alignment (see, for example, I. N. Shindyalov, et al., Protein Structure Alignment by Incremental Combinatorial Extension (CE) of the Optimal Path, 11 PROTEIN ENGINEERING 739-747 (1998), the disclosure of which is incorporated by reference herein), whereas 1BMG has a CE sequence identity of seven percent and an RMSD of 4.4 Angstroms with the protein domain of 1KGC(118-206) (which are the amino acid residues, by amino acid residue number, that comprise the domain, e.g., only a portion of the chain).

The A chain of 1G84 and the 1KGC domain show little sequence identity with each other or with the other proteins of the C1 set. These differences show up in the Fourier transforms. While the A chain of 1G84 and the 1KGC domain exhibit salient power at the frequencies 5 and 7 in their distance and exposure transforms, their relative amplitudes at frequency 5 are greater with respect to that at frequency 7, than exhibited by the other proteins of the C1 set. Despite these differences, the values of the correlation coefficients (for example, those listed in FIG. 16) are roughly parallel to what had been found for the globin proteins. In particular, the correlation coefficients devoid of the frequencies of hydrophobic prominent power amplitude and the range of frequencies of secondary structure, see “secondary eliminate” column of FIG. 5, described above, all range over statistically significant low level values as found for the globins.

While contributions to the hydrophobic spectral amplitude in the vicinity of a wavelength of two amino acid residues (as is expected for an amphipathic beta sheet) may be present, the ability to disentangle them from a random background depends upon their predominance. Such disentanglement has not been possible for the protein domains of 1IAK and 1KGC. The remaining seven structures exhibit a predominance of spectral amplitude in this region by comparison with thousands of randomization runs.

Protein 1BMG exhibits the greatest of such predominance. Its power amplitudes over the range of two to four amino acid residue wavelengths are shown in FIG. 20. FIG. 20 is a collection of spectral graphs illustrating percent Fourier power amplitudes of the sequence distance, exposure and hydrophobicity over the range of wavelengths from two to four amino acid residues, for the protein 1BMG. Pronounced amplitude is observed at a wavelength of two amino acid residues in all three spectra.

As observed for the globin proteins, there is also one greater range of fluctuating values in the background of the hydrophobicity spectrum over this range of wavelengths than is observed in the other two spectra. For 1BMG, the sum of the hydrophobic power amplitudes over the range of wavelengths from two to 2.6 amino acid residues is 40 percent greater than the average value obtained from thousands of runs where the amino acid sequence has been randomized.

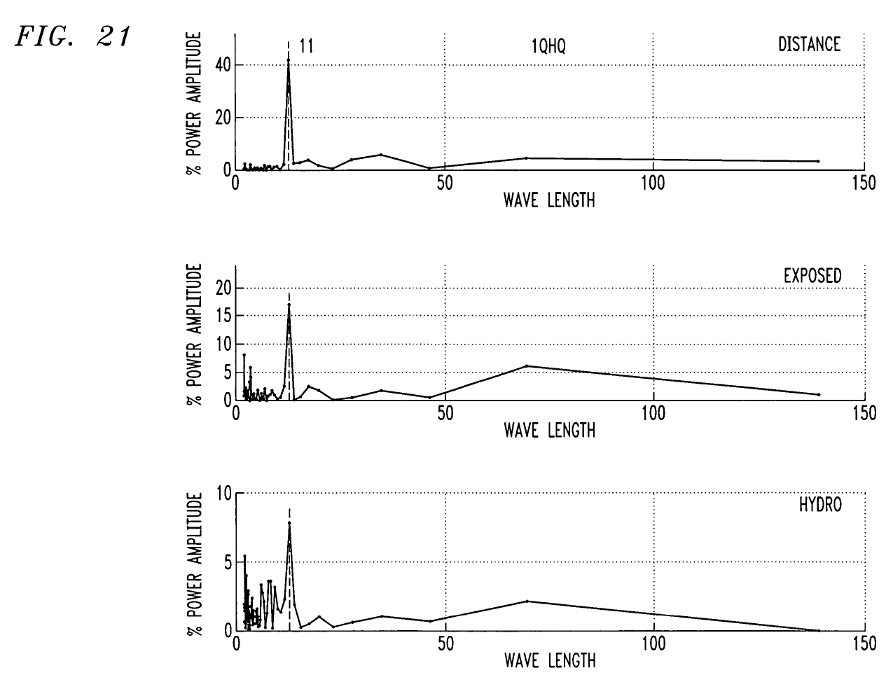

FIG. 21 is a collection of spectral graphs illustrating percent Fourier power amplitudes of the sequences of distance, exposure and hydrophobicity as a function of wavelength for the cuprodoxin protein 1QHQ. Of all the nine proteins of the plastocyanin/azurin set, this protein shows the sharpest single spectral correspondence, at frequency 11, across all three of the distributions.

Almost eight percent of the total power amplitude is found in this single hydrophobic spectral component. In contrast to the globin proteins and immunoglobulin proteins, this periodicity encompasses three different types of secondary structure, namely, helix, beta sheet and coil. This period with a 13-amino acid residue wavelength is also not easily visually apparent. The protein contains eight beta sheets. The prominent amplitude at frequency 11 includes traversal across these beta sheets as well as across two turns and the one major helix of the structure.

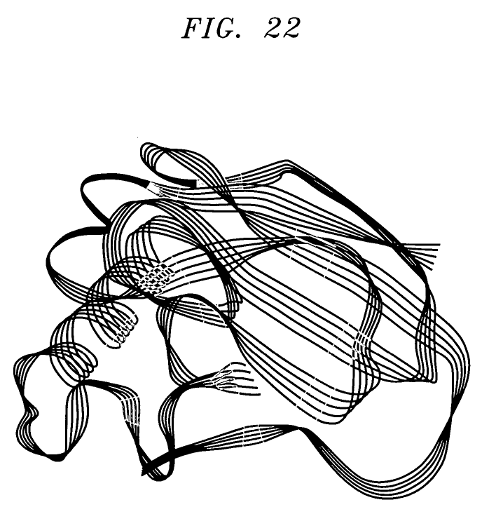

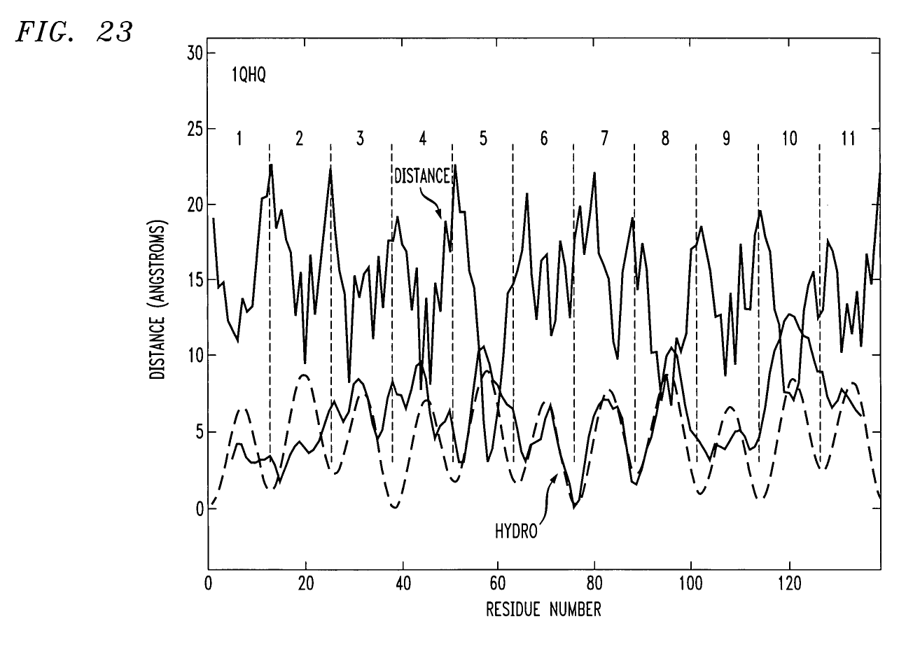

FIG. 22 is a ribbon diagram representation of the cuprodoxin protein 1QHQ. In FIG. 22, the amino acid residues at the local minima in each of the demarcated segments, 1 through 11, in the plot of distance with amino acid residue location, as will be described below in conjunction with the description of FIG. 23, are highlighted.

FIG. 23 is a spectral graph illustrating the values of ellipsoidal distance, from the centroid of the protein, with the hydrophobicity of the smoothed (shown as a connected line) and inverse transform of the prominent amplitudes (shown as a dashed line) for each amino acid residue along the sequence of the protein 1QHQ. The amino acid residues at the minimal distances in the demarcated segments 1, 6, and 7, are the amino acid residues in a turn, helix, and turn, respectively. With reference to the chart shown in FIG. 16, described above, reveals that the correlation coefficient, between the distances and the inverse transform of the amplitudes of the two prominent frequencies selected, 4 and 11, is 0.681. Further, the correlation coefficient between the distances and the inverse transform of the modes not selected, for example, those of frequencies 3 and 10, is 0.160.

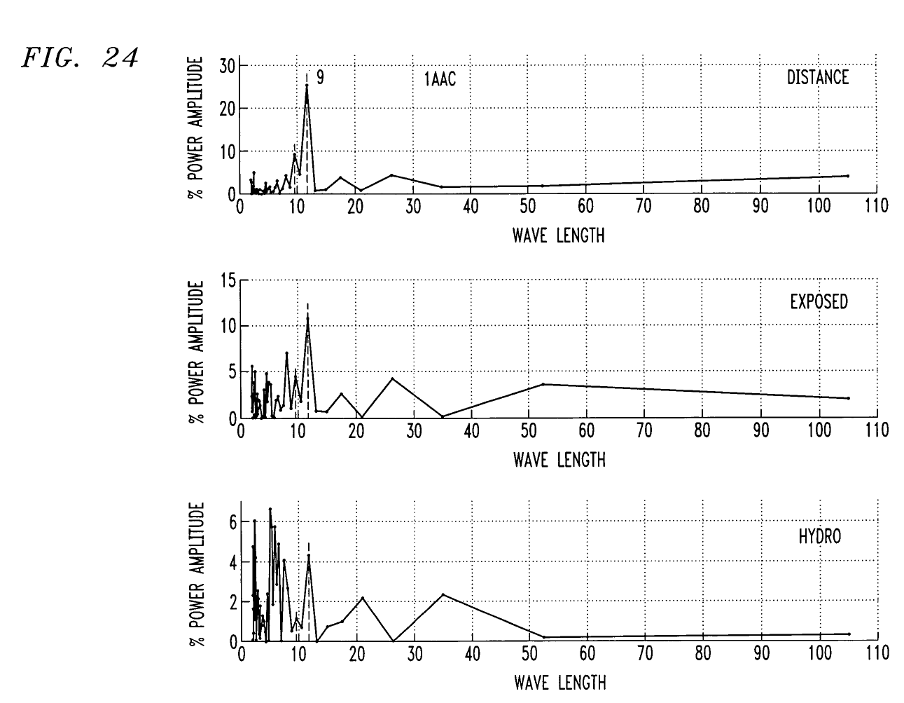

The cuprodoxin 1AAC also exhibits just two prominent frequencies, namely, 9 and 11 as shown in FIG. 24. FIG. 24 is a collection of spectral graphs illustrating percent Fourier power amplitudes of the sequences of distance, exposure and hydrophobicity, as a function of wavelength, for the protein 1AAC. Taking into account the difference in the numbers of amino acid residues of proteins 1AAC and 1QHQ, their modes of greatest power amplitude at frequencies 9 and 11, respectively, differ in wavelength by about two amino acid residues. Their smoothed values of amino acid residue hydrophobicity, however, correlate very differently with the distances from the protein interior.

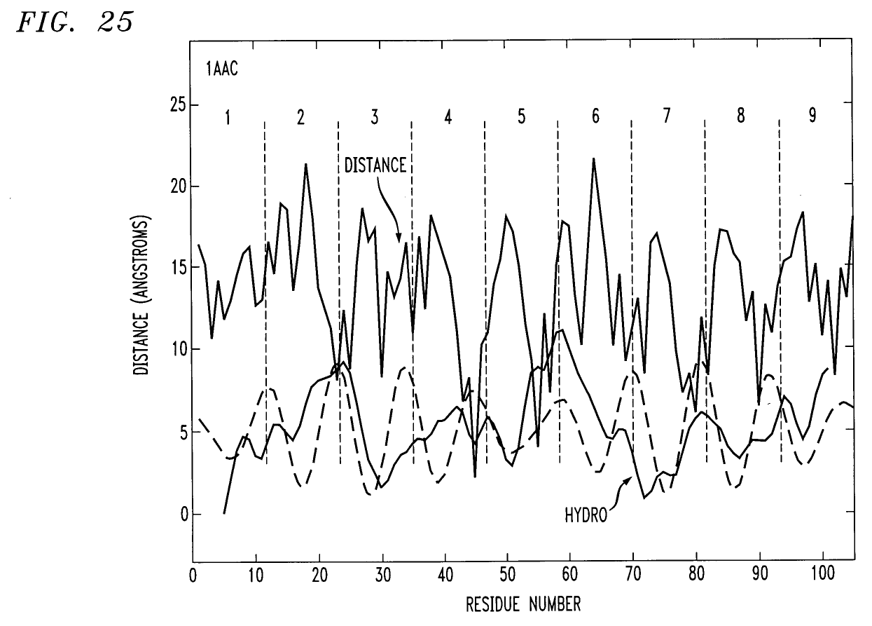

With reference to the chart shown in FIG. 16, described above, reveals that the smoothed distribution of hydrophobicity of 1AAC correlates marginally with the distances. The origin of this marginal correlation can be identified visually in FIG. 25, by examining the variation of distance and hydrophobicity with amino acid residue number, as shown. FIG. 25 is a spectral graph illustrating the values of ellipsoidal distance, from the centroid of the protein, with the hydrophobicity of the smoothed (shown as a connected line) and inverse transform of the prominent amplitudes (shown as a dashed line) for each amino acid residue along the sequence of the protein 1AAC.

To make a correlation, an increase in the value of amino acid residue hydrophobicity should be associated with a decrease in the value of distance from the protein interior. The overall increase of both the hydrophobicity and distance values within the demarcated segments 1 and 2 and a similar increase in demarcated segments 3 and 4 with a decrease in both values in segments 6 and 7 are notable.

Increased correlation is achieved between the hydrophobic inverse transform of the two prominent frequencies and the distances. This increased correlation is highlighted by the dashed line, which minimizes these in-step variations of both distributions with amino acid residue number, and sharpens the peak and valley registration. Minimizing the in-step variations and sharpening the peak and valley registration involved the elimination of the spectral range of hydrophobic wavelengths greater than that of frequency 9, among others, a range that does not provide correlation between the values of hydrophobicity and distances. This illustrates how variations in the distribution of hydrophobicity, presumably unrelated to the inside environment-to-outside environment excursions of the amino acid residues, can mask an underlying correlation that is present.

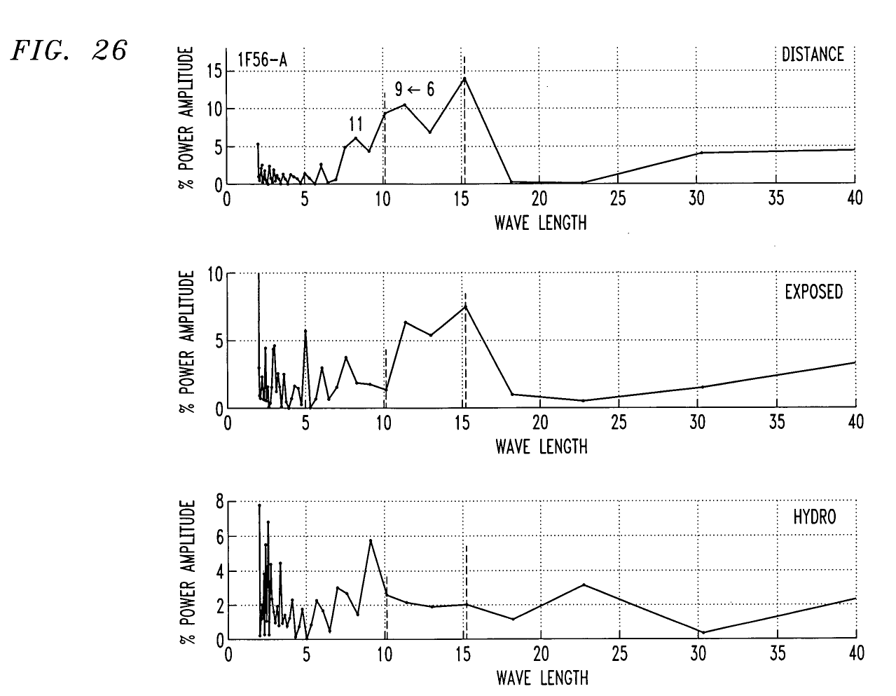

Prominent power amplitudes have comparable values over a broad range of adjacent frequencies of the A chain of the protein 1F56. This property is shown in FIG. 26. FIG. 26 is a collection of spectral graphs illustrating percent Fourier power amplitudes of the sequences of distance, exposure and hydrophobicity as a function of wavelength, for the A chain of protein 1F56. This broad distribution of power amplitudes is over the range of frequencies, 6 through 11.

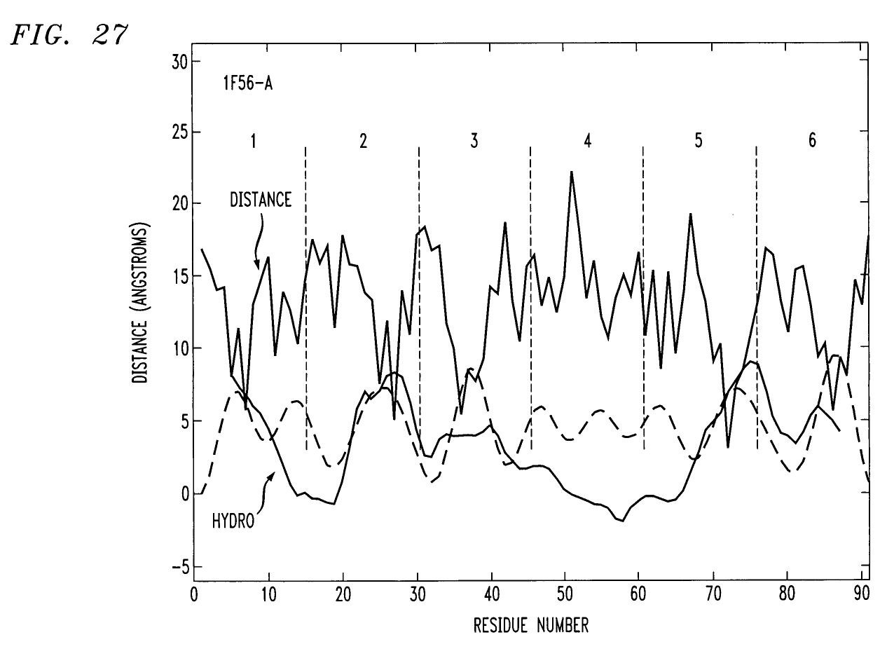

FIG. 27 shows the values of the inverse transform of the amplitudes over just this range of frequencies, 6 through 11 (with the exclusion of frequency 10), along with the smoothed profile and the distances. Namely, FIG. 27 is a spectral graph illustrating the values of ellipsoidal distance, from the centroid of the protein, with the hydrophobicity of the smoothed (shown as a connected line) and inverse transform of the prominent amplitudes (shown as a dashed line) for each amino acid residue along the sequence of the A chain of the protein 1F56.

The selection of this broad range of frequencies of comparable amplitude results in the reduced variation of the values of the inverse transform over the demarcated segments 3 to 5, a range of also minimal variation of the values of the smoothed profile. The greater registration of the peaks in the values of the inverse transform with the valleys in the distance profile, as compared with the smoothed values, are notable. This enhanced registration is also particularly apparent in the demarcated segments, 3 through 5, which is quantitatively expressed by the enhanced value of the hydrophobic inverse transform correlation coefficient with distance (as may be determined from a comparison of the “distance I-transform” and “distance smoothed” columns of the chart shown in FIG. 16 and as described above). In fact, this increase in correlation with respect to the smoothed hydrophobic values is found for all of the proteins listed in the chart shown in FIG. 16. For the globin proteins, such increase had been found for a majority of structures.

All nine cuprodoxins show a predominance of spectral amplitude over the range of two to 2.6 amino acid residue wavelengths, compared with what is found by randomizing the sequence. As with the immunoglobulins, the degree of this discrimination depends upon the extent of amphiphilicity of the beta sheets. For example, the A-chain of 1ADW shows a small four percent increase in spectral amplitude over a random background, while the A-chain of 1F56 shows a greater than 40 percent increase in spectral amplitude over a random background. Values for the remaining seven protein chains range between these values.

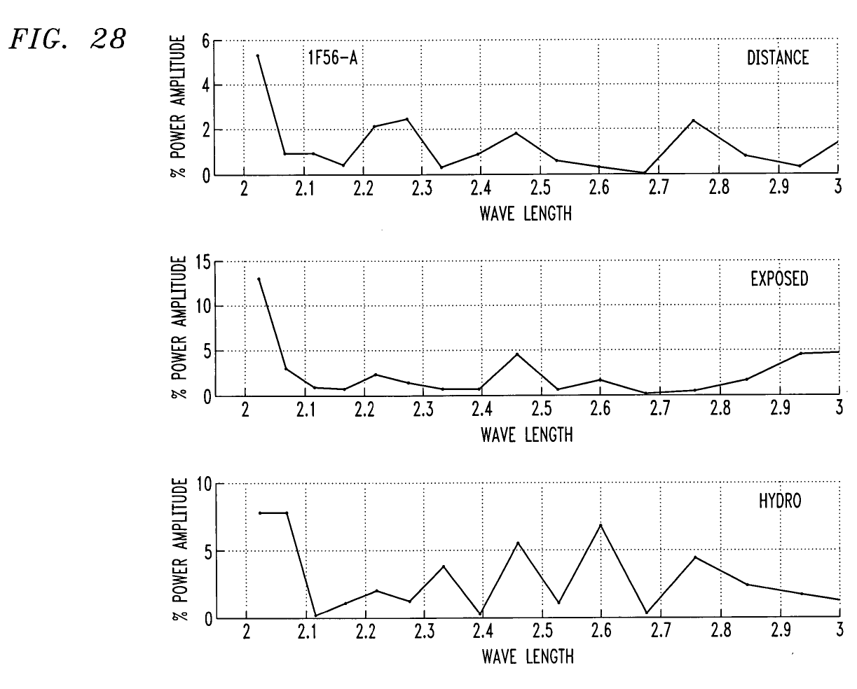

FIG. 28 shows the enhanced values of spectral amplitude for the A-chain of protein 1F56. Namely, FIG. 28 is a collection of spectral graphs illustrating percent Fourier amplitudes of the sequences of distance, exposure and hydrophobicity over the range of wavelengths from two to three amino acid residues, for the A chain of the protein 1F56.

Cysteine Proteinase Papain-Like Domains—Alpha and Beta Proteins:

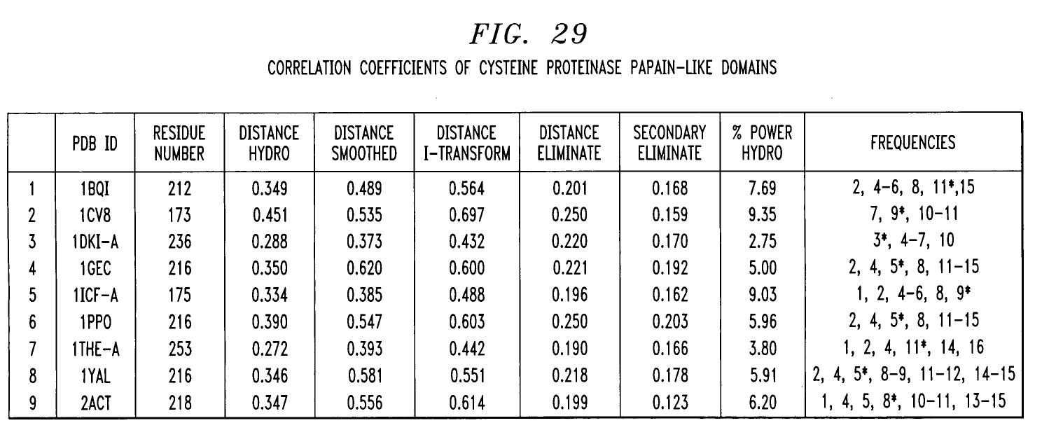

The cysteine proteinases exhibit an increase in amino acid residue number and structural complexity. Since the modes are now greater in number, it is expected that the percentage of total power amplitude in each mode to be reduced, compared with what had been obtained for the structures with fewer amino acid residues. This turns out to generally be the case, however, the prominent frequencies are now generally distributed over a broader range due to the greater structural complexity (for example, as can be seen from the chart shown in FIG. 29). FIG. 29 is a chart illustrating the correlation coefficients of cysteine proteinase papain-like domains. In particular, the column labeled “frequencies,” which lists the prominent frequencies identified from the Fourier transform of the distances, illustrates that the prominent frequencies are now generally distributed over a broader range.

As can be seen from FIG. 29, the two structures with the fewest number of amino acid residues of the nine, 1CV8 and the A chain of 1ICF, differ in structural detail and sequence, for example, having a CE RMSD of 3.6 Angstroms and a sequence identity of 13 percent. These differences in structure and sequence are reflected in the Fourier spectra. The most prominent periodicity of these two structures is 9 fold, with a wavelength of approximately 19 amino acid residues in extent.

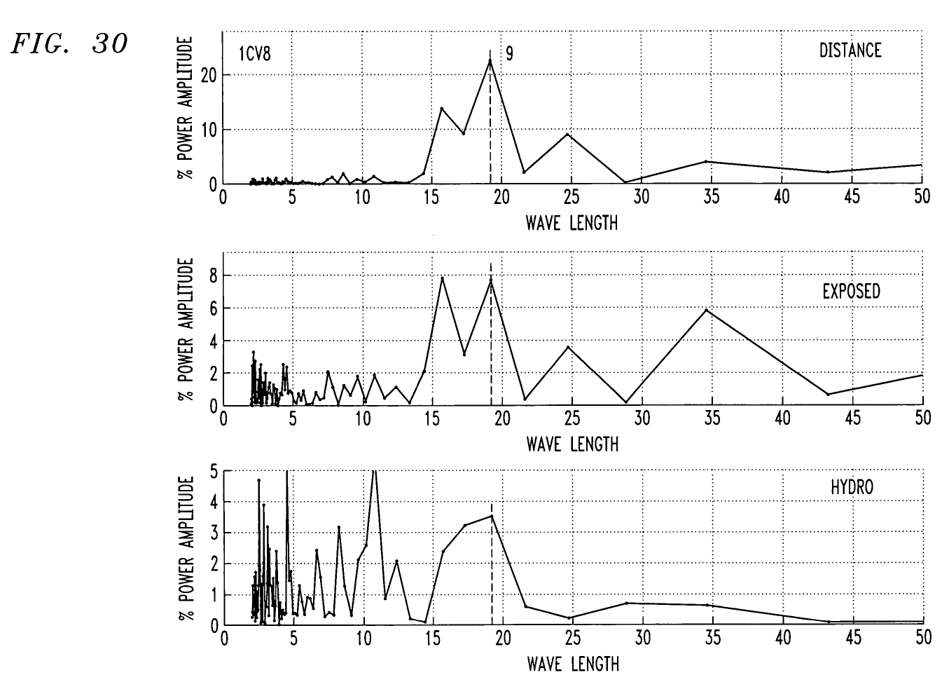

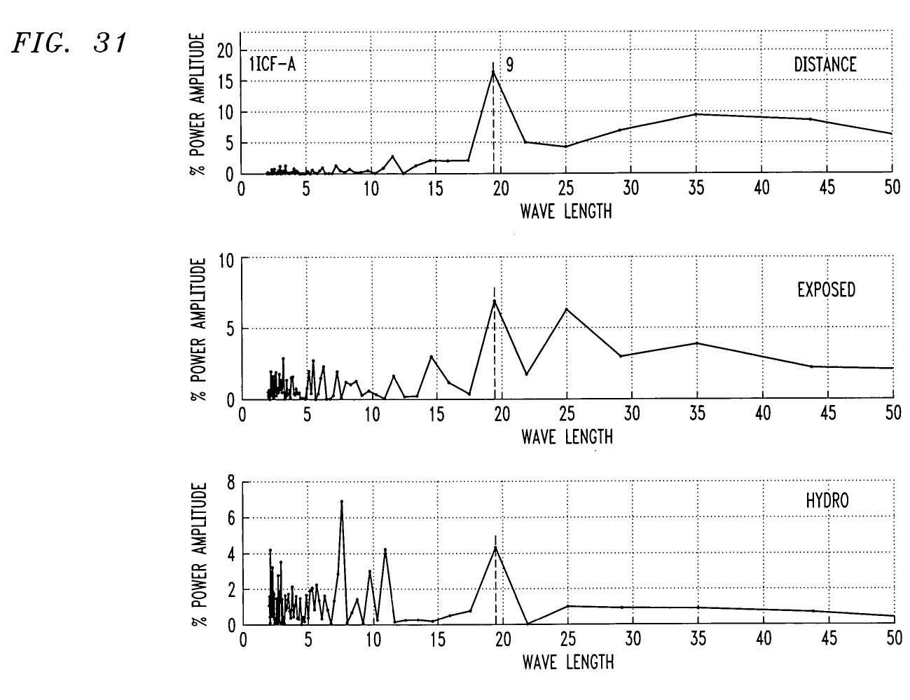

The percentages of the power in the distance spectra of 1CV8 and the A chain domain of 1ICF at this frequency, e.g., shown in the topmost frames of FIGS. 30 and 31, described below, are 22.5% and 16.5%, respectively. Namely, FIG. 30 is a collection of spectral graphs illustrating percent Fourier power amplitudes of the sequences of distance, exposure and hydrophobicity as a function of wavelength, for the protein domain 1CV8. FIG. 31 is a collection of spectral graphs illustrating percent Fourier power amplitudes of the sequences of distance, exposure and hydrophobicity as a function of wavelength, for the A chain of the protein 1ICF.

From FIG. 30, it is notable that the enhanced amplitudes in the distance spectra of 1CV8 at frequencies 9, 10 and 11, are mirrored by the correspondingly enhanced amplitudes at these same frequencies in the hydrophobicity and exposure spectra. FIG. 31 shows that comparable values of power amplitude are not observed at frequencies 10 and 11 in all three spectra of the 1ICF domain.

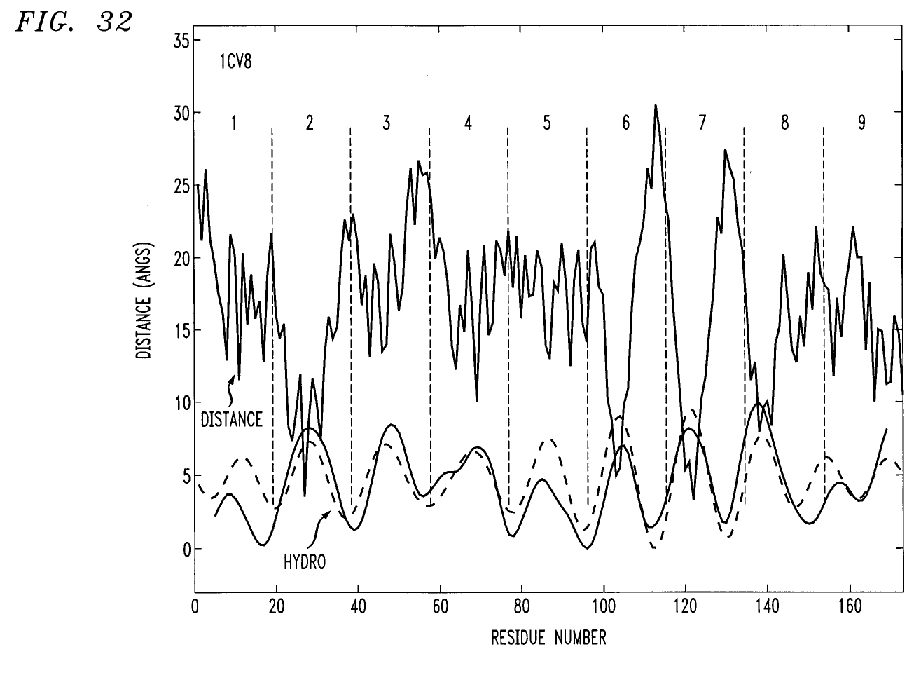

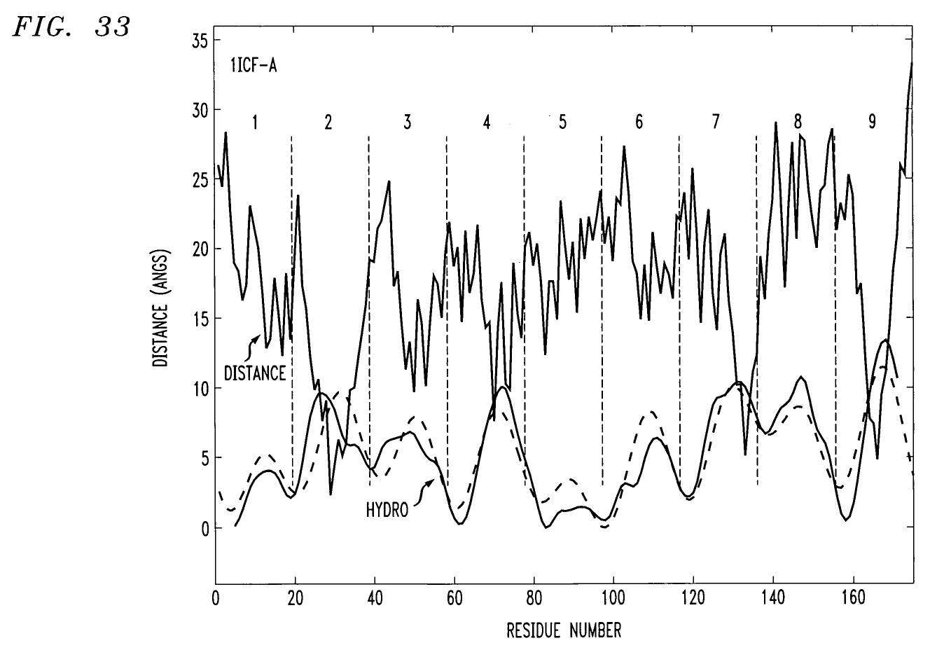

The distances smoothed, and the inverse transform hydrophobicities of the amino acid residues of the 1CV8 and 1ICF domains are shown in FIGS. 32 and 33, described below, respectively. FIG. 32 is a spectral graph illustrating the values of ellipsoidal distance, from the centroid of the protein, along with the hydrophobicity of the smoothed (shown as a connected line) and the inverse transform of the prominent amplitudes (shown as a dashed line) for each amino acid residue along the sequence of the protein 1CV8. FIG. 33 is a spectral graph illustrating the values of ellipsoidal distance, from the centroid of the protein with the hydrophobicity of the smoothed (shown as a connected line) and inverse transform of the prominent amplitudes (shown as a dashed line) for each amino acid residue along the sequence of the A chain of the protein 1ICF.

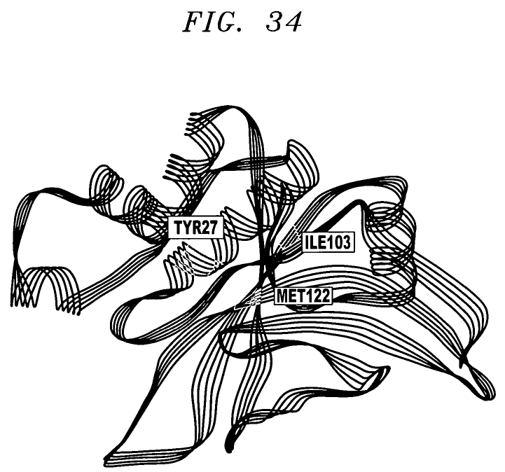

The peaks in the values of hydrophobicity register with the minimal distances from the protein centroid for both the 1CV8 and 1ICF domains, however, differences in the spectra of the two proteins are evident. The nine-fold periodicity of the inverse transform and smoothed profiles of 1CV8 is more pronounced than that observed for 1ICF. The nine-fold periodicity of 1CV8 encompasses the three most interior amino acid residues, TYR27, ILE103 and MET122, which are found in a helix and two beta sheets, respectively. The location of these amino acid residues in the 1CV8 protein is given in FIG. 34. Namely, FIG. 34 is a ribbon diagram illustrating the location of the three most interior amino acid residues, TYR27, ILE103 and MET122, of the nine-fold prominent period of the protein 1CV8.

The percent power amplitude in this single nine-fold prominent period of the protein 1CV8 is 3.5 percent of the total hydrophobic power amplitude. The inverse transform of the amplitude of this single frequency yields a correlation coefficient with a distance of 0.471. Reference to the chart shown in FIG. 29, described above, reveals that all four frequencies of prominent amplitude yield a correlation coefficient of 0.697, which is yet another example, as was the case for the cuprodoxins, of a hydrophobic periodicity that correlates with structure, involves amino acid residues in different secondary structures, is not involved in a structure of any particular symmetry and is not visually apparent.

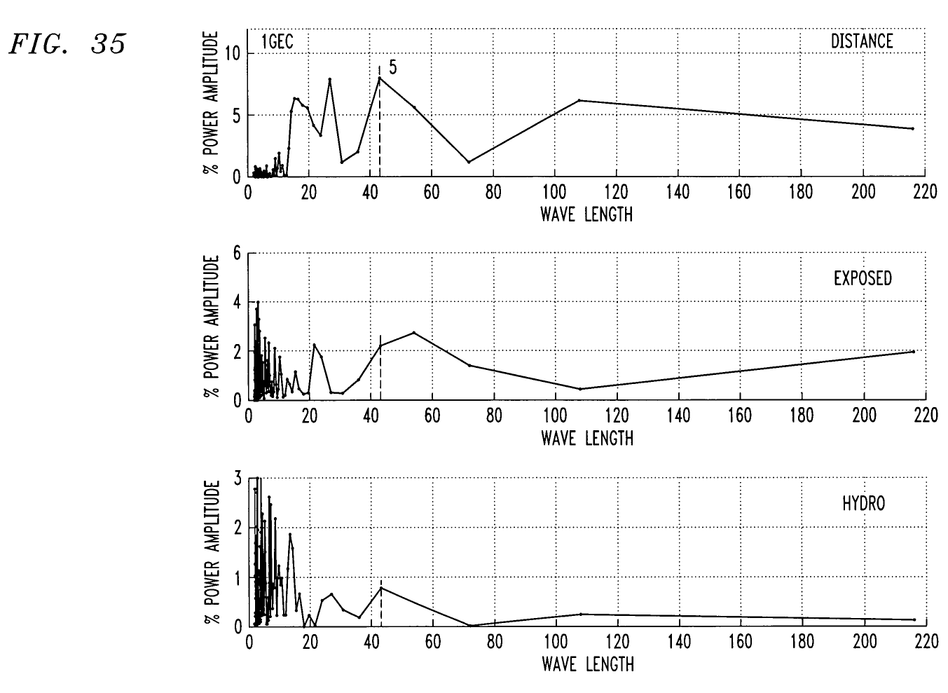

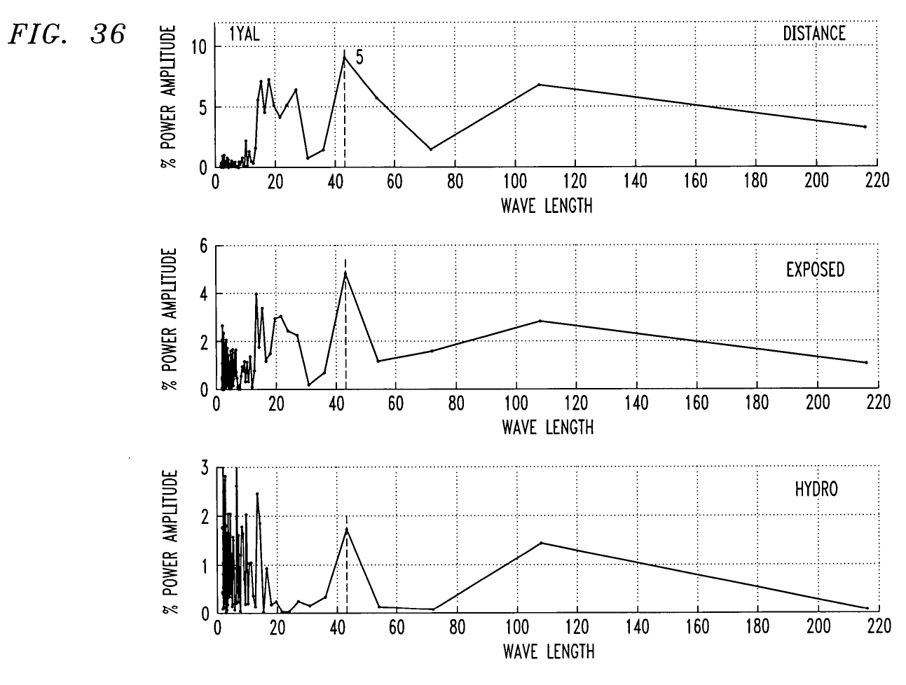

The protein domains, 1GEC and 1YAL, have a CE RMSD of 1.1 Angstroms and a sequence identity of 69.4 percent. The percent power amplitudes for the 1GEC and 1YAL proteins are shown in FIGS. 35 and 36, described below, respectively. FIG. 35 is a collection of spectral graphs illustrating percent Fourier power amplitudes of the sequences of distance, exposure and hydrophobicity as a function of wavelength, for the protein 1GEC. FIG. 36 is a collection of spectral graphs illustrating percent Fourier power amplitudes of the sequences of distance, exposure and hydrophobicity as a function of wavelength, for the protein 1YAL.

The ordinate scales in FIGS. 35 and 36 have been chosen to be the same to facilitate comparison. Comparable distances yield comparable values of the power amplitudes of distance shown in the uppermost frames of each of FIGS. 35 and 36. The prominent frequencies (for example, those listed in the “frequencies” column of FIG. 29, described above) also differ only slightly. The relative values of the power amplitudes of hydrophobicity of the two proteins also track each other, but not as closely as do the distances.

The percentage of the total hydrophobic power of frequency 5 of 1YAL is roughly twice the value for 1GEC. Differences are also noted in the relative power amplitudes at other wavelengths of the hydrophobicity spectra. These differences translate into differences in the values of the correlation coefficients of these proteins (see, for example, the chart shown in FIG. 29, described above).

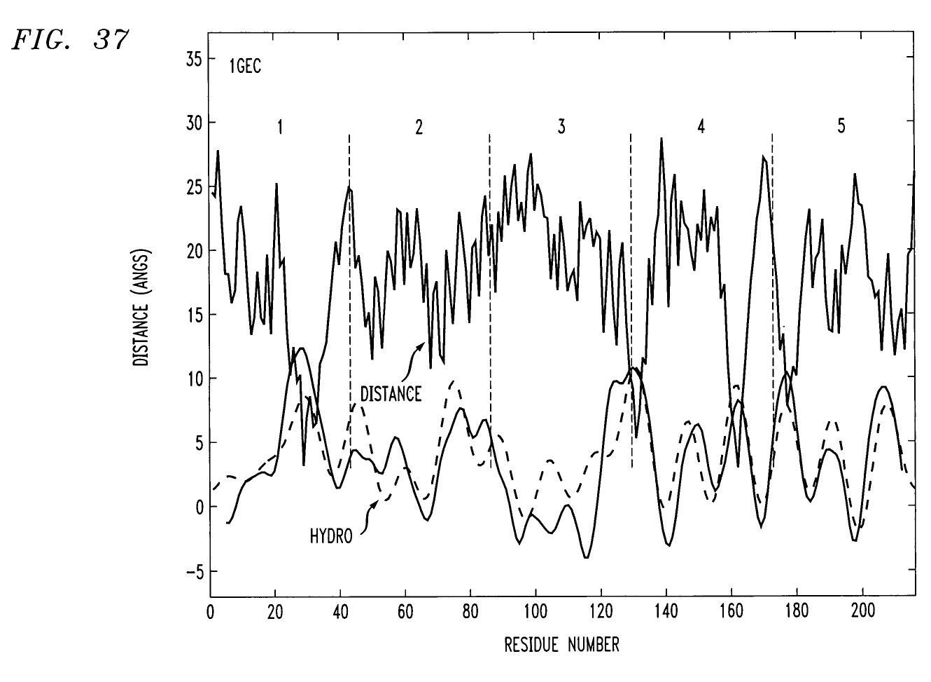

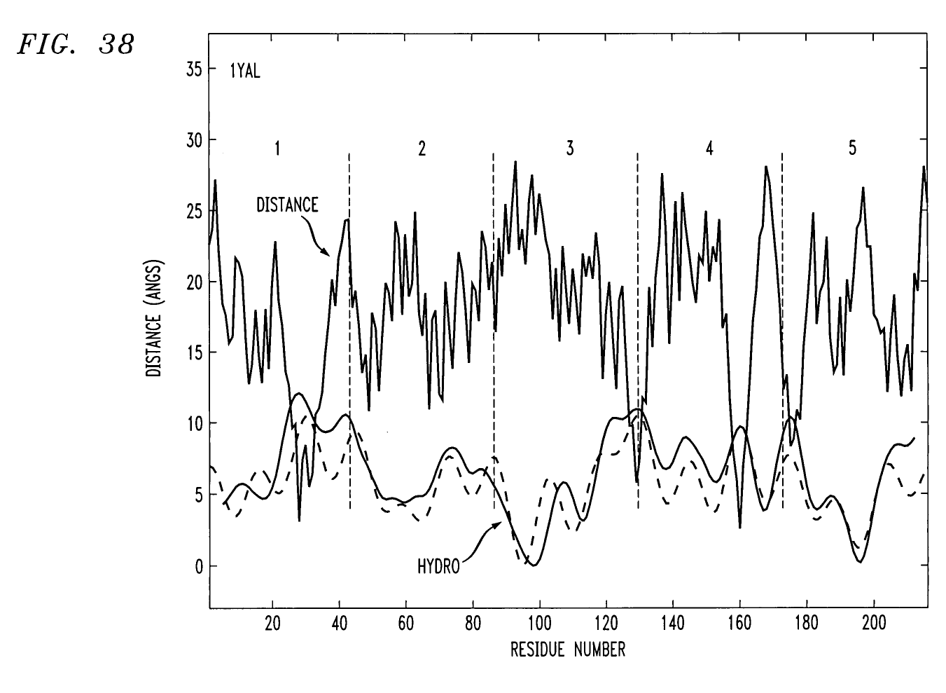

FIG. 37 is a spectral graph illustrating the values of ellipsoidal distance, from the centroid of the protein, along with the hydrophobicity of the smoothed (shown as a connected line) and inverse transform of the prominent amplitudes (shown as a dashed line) for each amino acid residue along the sequence of the protein 1GEC. FIG. 38 is a spectral graph illustrating the values of ellipsoidal distance, from the centroid of the protein, with the hydrophobicity of the smoothed (shown as a connected line) and inverse transform of the prominent amplitudes (shown as a dashed line) for each amino acid residue along the sequence of the protein 1YAL.

While the smoothed hydrophobicity and inverse transform values of 1GEC and 1YAL (see, for example, FIGS. 37 and 38) do not track each other as closely as the distances, their overall structure exhibits considerable similarity. Furthermore, registration between the peaks and valleys of the respective hydrophobicities and distances is achieved with, at most, six percent of the total hydrophobic spectral power.

Since helical secondary structure is present in the papain-like domains, a spectral range of two to five amino acid residue wavelengths has been examined to determine if spectral amplitude is shifted out of this frequency range upon randomization of amino acid residue hydrophobicity along the chain. Since the nine papain-like domains are more heterogeneous in structure than the globin proteins, it is perhaps not surprising that the results found are somewhat different. Thousands of randomization runs for the five protein domains, namely the A chain of 1DKI, 1GEC, the A chain of 1THE, 1YAL, and 2ACT show an average reduction from the native sequence of eight percent or greater in percentage power amplitude over this range. The three proteins 1BQI, 1CV8 and 1PPO show minimal change, while the A chain of the protein 1ICF actually shows an eight percent increase. Further investigation of the origin of such differences is, therefore, of interest.

It is further interesting to note that the registration between the peaks of the smoothed hydrophobicity distributions and inverse transform values with the valleys of the distances can be used as a check or validation of predicted protein structures. Further, since regions of the entire protein chain are displayed, particular regions where the predicted structure appears to be problematic can be identified.

Regarding protein design, amphiphilic helical secondary structures can be built by suitably choosing the sequence of amino acid residue hydrophobicity. The present analysis, relating hydrophobic sequence periodicity to a feature of three-dimensional protein structure suggests a strategy for choosing this periodicity in a way that would be consistent with the tertiary protein structure desired. This strategy in choosing the periodicity would necessitate the simultaneous optimization of sequence hydrophobicity for secondary structure, as well as for the distribution of amino acids from the protein interior environment-to-exterior environment.

Although illustrative embodiments of the present invention have been described herein, it is to be understood that the invention is not limited to those precise embodiments, and that various other changes and modifications may be made by one skilled in the art without departing from the scope or spirit of the invention.

What is claimed is:

1. A method of characterizing at least a portion of a protein structure comprising amino acid residues, the method comprising the steps of:
determining a set of values characterizing the protein structure, wherein each value represents a distance from a center of the protein structure to a center of a given one or more of the amino acid residues;
obtaining one or more other sets of values characterizing a hydrophobicity of the protein structure;
performing a Fourier transform on each of the sets of values to obtain transformed value sets; and
comparing the transformed value sets to correlate the hydrophobicity with the protein structure.

2. The method of claim 1, wherein the one or more other sets of values characterizing the hydrophobicity of the protein structure comprise either solvent exposure values for the given one or more of the amino acid residues, hydrophobicity values for the given one or more of the amino acid residues or both solvent exposure and hydrophobicity values for the given one or more of the amino acid residues.

3. The method of claim 1, wherein the distance from the center of the protein structure to the center of the given one or more of the amino acid residues is determined using an ellipsoidal distance metric.

4. The method of claim 1, wherein the center of the protein structure comprises a centroid of the protein structure.

5. The method of claim 1, wherein the center of the protein structure is determined based on the center of each of the amino acid residues making up the protein.

6. The method of claim 1, wherein the center of each of the given one or more amino acid residues comprises a centroid of the amino acid residue.

7. The method of claim 1, wherein the transformed value sets are compared to correlate the hydrophobicity with features of the protein structure.

8. The method of claim 7, wherein the features comprise major structural motifs.

9. The method of claim 1, further comprising the steps of:
extracting values from the one or more other sets of values characterizing the hydrophobicity of the protein structure that correlate with features of the protein structure; and
performing an inverse transform of the extracted values.

10. The method of claim 1, further comprising the steps of:
extracting values from the one or more other sets of values characterizing the hydrophobicity of the protein structure that correlate with features of the protein structure; and
performing window averaging and smoothing of the extracted values.

11. The method of claim 1, wherein the transformed value sets are compared visually.

12. The method of claim 1, wherein correlation coefficients are used to compare the transformed value sets.

13. The method of claim 1, further comprising the step of window averaging one or more values in the set of values characterizing the protein structure

14. An apparatus for characterizing at least a portion of a protein structure comprising amino acid residues, the apparatus comprising:
a memory; and
at least one processor, coupled to the memory, operative to:
determine a set of values characterizing the protein structure, wherein each value represents a distance from a center of the protein structure to a center of a given one or more of the amino acid residues;
obtain one or more other sets of values characterizing a hydrophobicity of the protein structure;
perform a Fourier transform on each of the sets of values to obtain transformed value sets; and
compare the transformed value sets to correlate the hydrophobicity with the protein structure.

15. The apparatus of claim 14, wherein the one or more other sets of values characterizing the hydrophobicity of the protein structure comprise either solvent exposure values for the given one or more of the amino acid residues, hydrophobicity values for the given one or more of the amino acid residues or both solvent exposure and hydrophobicity values for the given one or more of the amino acid residues.

16. The apparatus of claim 14, wherein the at least one processor is further operative to:
extract values from the one or more other sets of values characterizing the hydrophobicity of the protein structure that correlate with features of the protein structure; and
perform an inverse transform of the extracted values.

17. The apparatus of claim 14, wherein the at least one processor is further operative to:
extract values from the one or more other sets of values characterizing the hydrophobicity of the protein structure that correlate with features of the protein structure; and
perform window averaging and smoothing of the extracted values.

18. An article of manufacture for characterizing at least a portion of a protein structure comprising amino acid residues, comprising a machine readable medium containing one or more programs which when executed implement the steps of:
determining a set of values characterizing the protein structure, wherein each value represents a distance from a center of the protein structure to a center of a given one or more of the amino acid residues;
obtaining one or more other sets of values characterizing a hydrophobicity of the protein structure;
performing a Fourier transform on each of the sets of values to obtain transformed value sets; and
comparing the transformed value sets to correlate the hydrophobicity with the protein structure.

19. The article of manufacture of claim 18, wherein the one or more other sets of values characterizing the hydrophobicity of the protein structure comprise either solvent exposure values for the given one or more of the amino acid residues, hydrophobicity values for the given one or more of the amino acid residues or both solvent exposure and hydrophobicity values for the given one or more of the amino acid residues.

20. The article of manufacture of claim 18, further comprising the steps of:
extracting values from the one or more other sets of values characterizing the hydrophobicity of the protein structure that correlate with features of the protein structure; and
performing an inverse transform of the extracted values.

21. The article of manufacture of claim 18, further comprising the steps of:
extracting values from the one or more other sets of values characterizing the hydrophobicity of the protein structure that correlate with features of the protein structure; and
performing window averaging and smoothing of the extracted values.
